(19) United States
(12) Patent Application Publication
(10) Pub. No.: US 2026/0193219 A1
(43) Pub. Date: Jul. 9, 2026

(54) TRIAZINE DERIVATIVES FOR TREATING DISEASES RELATING TO NEUROTROPHINS

(71) Applicant: AlzeCure Pharma AB, Huddinge (SE)

(72) Inventors: Gunnar Nordvall, Rònninge (SE); Pontus Forsell, Huddinge (SE)

(21) Appl. No.: 19/548,423

(22) Filed: Feb. 24, 2026

Related U.S. Application Data
(63) Continuation of application No. 17/633,694, filed on Aug. 28, 2020, now abandoned.

(30) Foreign Application Priority Data
Aug. 29, 2020 (GB) 1912404.9

(51) Int. Cl. C07D 251/34; A61P 25/14; A61P 25/18; A61P 25/24; A61P 25/28

(52) U.S. Cl. CPC C07D 251/34; A61P 25/14; A61P 25/18; A61P 25/24; A61P 25/28

(57) ABSTRACT

There is provided a compound of formula I,

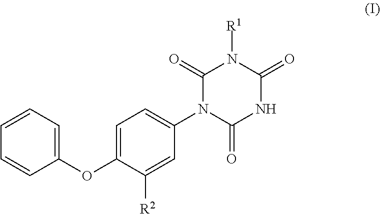

wherein R¹ and R² are as defined herein, which compounds are useful in the treatment of diseases characterised by impaired signalling of neurotrophins and/or other trophic factors, such as Alzheimer's disease and the like.

### CROSS-REFERENCE TO RELATED APPLICATIONS

This application is a Continuation Application of U.S. application Ser. No. 17/633,694, filed on Feb. 8, 2022, which is the U.S. national stage filing, under 35 U.S.C. § 371(c), of International Application No. PCT/GB2020/052068, filed on Aug. 28, 2020, which claims priority to United Kingdom Patent Application No. 1912404.9, filed on Aug. 29, 2019.

### FIELD OF THE INVENTION

This invention relates to novel pharmaceutically-active compounds, to pharmaceutical compositions comprising such compounds, as well as to their pharmaceutical use. In particular, the invention relates to the use of these compounds and compositions in methods for the treatment and/or prevention of diseases characterised by impaired signalling of neurotrophins and/or other trophic factors.

### BACKGROUND OF THE INVENTION

The listing or discussion of an apparently prior-published document in this specification should not necessarily be taken as an acknowledgement that the document is part of the state of the art or is common general knowledge.

Nerve growth factor (NGF), Brain Derived Neurotrophic Factor (BDNF), neurotrophin-3 (NT-3) and neurotrophin-4/5 all belong to the neurotrophin protein family. These hormones act through a class of receptor tyrosine kinases called tropomyosin-receptor kinase (Trk). Ligand binding to Trks initiates receptor dimerization and autophosphorylation of the kinase domain, which activates the kinase activity of the receptor. This results in further receptor phosphorylation at Tyr490, Tyr751 and Tyr785 of TrkA (or their equivalent residues in other Trk receptors). This phosphorylation leads to adaptor binding sites that couple the receptor to SHC adaptor protein 1 (SHC-1), phosphoinositide 3-kinase (PI3K) and phospholipase Cγ1 (PLCγ1). The coupling of adaptor proteins to the receptor initiates several different cellular events leading to e.g. neurite outgrowth and axonal elongation. These receptors, and their signalling pathways, play a pivotal role in many key processes in the brain e.g. hippocampal neurogenesis, synaptic plasticity, and long-term potentiation, a proposed mechanism underlying memory formation at the level of the synapse. Both NGF/TrkA and BDNF/TrkB-stimulated signalling is also necessary for the survival and morphogenesis of neurons.

In addition to activation of Trk-receptors by classical ligand binding, there are ligand independent events that can regulate neurotrophin signalling.

The balance between the activity of the receptor tyrosine kinase and the activity of tyrosine phosphatases intricately regulates the levels of phosphorylated receptor. Thus, protein tyrosine phosphatases such as PTP-1B or other phosphatases can increase neurotrophin signalling and regulate temporal and spatial activity of the Trk-receptor as well as receptor tyrosine kinases.

Also, adenosine and adenosine agonists can mediate phosphorylation of Trk-receptors, via a mechanism that requires the adenosine 2A (A2A) receptor. This phosphorylation of Trk-receptors is independent of ligand binding suggesting that modulation of Trk-receptor signalling can be accomplished by several different mechanisms.

Synapse loss and a decrease in the hippocampal volume are pathological signatures of Alzheimer's disease in the brain and a number of studies suggest that synapse loss is the best neuroanatomical indicator of cognitive decline in the disease. Basal forebrain cholinergic neurons (BFCN) are a subpopulation of neurons that seem to be particularly vulnerable to the pathology of AD. Dysfunctional atrophy of these neurons, which in turn results in severe loss of cortical and hippocampal innervation, may be the source for the malfunction of the cholinergic system in AD (Bartus R T Exp Neurol 2000; 163:495-529). The severe cortical cholinergic deficits in the disease also include a loss of choline acetyltransferase (ChAT) and acetylcholinesterase (AChE) activity. The basal forebrain cholinergic system is dependent on NGF and cholinergic basal forebrain neurons are the major cell group that expresses the receptor for NGF, i.e. TrkA. Although the role of NGF in cholinergic neuronal survival and function is well established, studies have also shown neuroprotective/neurorestorative effects mediated by this system, e.g. that axotomized cholinergic projections in animals can be rescued by TrkA activation (Lucidi-Phillipi C A, Neuron., 1996, 16(3):653-663).

An early morphological change in the brain of AD-patients is a decreased hippocampal volume. BDNF/TrkB-stimulated signalling has previously been shown to be necessary for survival and morphogenesis of especially hippocampal neurons. Moreover, it is widely accepted that BDNF plays a critical role in neuronal plasticity and long-term potentiation (LTP). Indeed, a growing body of experimental evidence suggests that increased BDNF signalling could potentially improve cognition in AD. The transplantation of stem cells into the brain of a triple-transgenic mouse model of AD, that expresses amyloid and tau pathology, i.e. the major neuropathological hallmarks of AD, results in improved cognition (Blurton-Jones M, PNAS, 2009. 106(32): p. 13594-13599). This effect is mediated by BDNF as gain-of-function studies show that recombinant BDNF mimics the beneficial effects of neural stem cell (NSC) transplantation. Furthermore, loss-of-function studies show that depletion of NSC-derived BDNF fails to improve cognition or restore hippocampal synaptic density.

Given the potent neuroprotective and neurorestorative effects of the TrkA/NGF and TrkB/BDNF systems, small molecule positive modulators of neurotrophin signalling might be beneficial in treating a number of diseases with neurodegeneration including, but not limited to, Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and other parkinsonian disorders. The modulators can also be used in the treatment of diseases where enhancement of nerve regeneration is beneficial, such as demyelinating diseases including, but not limited to, multiple sclerosis. The modulators could also be used for neuroprotection before or after an insult such as spinal cord injury, stroke, hypoxia, ischemia, brain injury including traumatic brain injury. Moreover, the important role of these neurotrophin systems in synaptic plasticity is thought to mediate learning and memory processes, and indicates that the modulators could also be used in disorders where cognitive function is impaired, including, but not limited to, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia.

Recent data have also indicated that NGF/TrkA and BDNF/TrkB systems may operate as metabotrophins, that is, be involved in the maintenance of cardiometabolic homeostasis (glucose and lipid metabolism as well as energy balance, cardioprotection, and wound healing) (Chaldakov G, Arch. Ital. Biol. 2011 June 149(2):257-63). In fact, mutations in the genes encoding BDNF and its receptor TrkB have been shown to lead to severe obesity in humans (Yeo, G S. et al. Nat. Neurosci. 2004, 7, 1187-1189). Therefore, indications such as atherosclerosis, obesity, diabetes and metabolic syndrome could also benefit from NGF/TrkA and BDNF/TrkB directed therapies.

Another area of interest when it comes to neurotrophin signalling is neuropsychiatric disorders (Castrén E et al., Neurobiol Dis. 2016 Jul. 15, 30169-3). Studies have, for example, clearly demonstrated that depressed patients have reduced serum BDNF levels, which are restored after successful recovery (Shimizu et al., 2003, Sen et al., 2008). Moreover, several studies have demonstrated that chronic treatment with various antidepressant drugs increase BDNF mRNA and protein levels in the cerebral cortex and hippocampus (Calabrese et al., Psychopharmacology, 2011, 215, pp. 267-275). Also, local administration of BDNF into the brain has been shown to reduce depression-like behavior and mimic the effects of antidepressants (Hoshaw et al., Brain Res., 2005, 1037, pp. 204-208). Notably, the role for BDNF does not seem to be restricted to depression; it has also been implicated in other disorders, such as anxiety and schizophrenia (Castren E., Handb. Exp. Pharmacol., 2014, 220, pp. 461-479). These data suggest that therapies targeting neurotrophin systems e.g. NGF/TrkA and BDNF/TrkB could have a therapeutic effect in several neuropsychiatric disorders, including, but not limited to, depression, schizophrenia and anxiety.

It has also been demonstrated that both BDNF and NT3 stimulate gastrointestinal motility, accelerate colonic transit time and relieve constipation in humans (Coulie B., et al., Gastroenterology, 2000, 119(1), 41-50). The levels of BDNF in colonic biopsies from patients with slow-transit constipation have also been found to be reduced compared to healthy controls (Chen et al., Acta. Physiol., 2014, 212(3), 226-238). Furthermore, one of the observed adverse events in a large phase 3 clinical trial with >1,000 patients with the neurological disorder amyotrophic lateral sclerosis undergoing treatment with recombinant BDNF was increased gut motility, diarrhoea and relief of constipation (Neurology, 1999, 52(7), 1427). A similar observation was also made in a smaller clinical trial of BDNF in patients with diabetic neuropathy (Wellmer A. et al., J. Peripher. Nerv. Syst., 2001, 6(4), 204-210). Preclinical models have also suggested that BDNF and NT3 may play a role in the regulation of gastrointestinal motility. BDNF+/− heterozygous mice display decreased stool frequency and increased total gastrointestinal transit time, demonstrating that lower BDNF levels reduces gastrointestinal motility. BDNF also relieved loperamide-induced constipation in mice (Chen et al., Acta. Physiol., 2014, 212(3), 226-238). Accordingly, it is believed that neurotrophins and their receptors may play an important role in maintaining normal motility and therefore that modulation of neurotrophin signaling could represent a promising strategy for improving gut motility in patients suffering from constipation.

The finding that NGF and BDNF play important roles in neuronal homeostasis in combination with their neuroprotective and neurorestorative effect makes these pathways highly suitable as candidates for drug intervention for the treatment of diseases of the central nervous system and the peripheral nervous system. However, BDNF and NGF are themselves not ideal drug candidates due to their pharmacokinetic properties, the difficulties in administration and their limited ability to cross the blood-brain barrier. This has led to several attempts to identify peptides, cyclized peptides, peptide mimetics, small molecule agonist or selective modulators of NGF or BDNF. Several natural products such as gambogic amide (and analogues thereof), deoxygedunin and 7,8-dihydroxyflavone have been demonstrated to act as TrkA or TrkB agonists. Moreover, the tricyclic depressant amitriptyline has also been shown to be a TrkA and TrkB agonist. However, there is currently no specific TrkA or TrkB agonist that has reached the market. Therefore, there is an unmet need in the art for small molecule compounds that have the ability to stimulate or modulate TrkA and/or TrkB receptors, in combination with TrkC, FGFR1 and/or IGF1R and optionally other receptor tyrosine kinases for the treatment of both neurological and non-neurological disorders. There is still a need for compounds that have an improved potency and improved selectivity to TrkA and/or TrkB receptor.

BDNF production can be affected by a polymorphism within the BDNF gene (rs6265) causes a valine (Val) to methionine (Met) substitution at codon 66 (Val66Met). This polymorphism is found in approximately 30% of Caucasians and up to 70% in Asian populations. The presence of one or two Met alleles is associated with lower BDNF production in a subject. This lower BDNF production can lead to increased cognitive decline and decreased hippocampal volume.

A study by Boots et al (Neurology, 2017, 88, 1-9) demonstrated that subjects suffering sporadic Alzheimer's disease who carry the BDNF Met allele experience a steeper decline in episodic memory and executive function than non-carriers. Greater memory decline and decreased hippocampal function have also been observed in Val66Met patients with familial Alzheimer's disease (Lim et al., Brain, 2016, 139(10), 2766-2777). The same study also showed increased tau-protein and phosphorylated tau-protein in the cerebrospinal fluid in this patient group. The decline in memory in subjects with pre-clinical or clinical Alzheimer's disease was exacerbated by greater amyloid plaque burden, thus suggesting that it is possible to treat Alzheimer's disease at various stages of the disease by potentiating the effects of BDNF in patients with the Val66Met polymorphism. Such treatment may lead to neuroprotection and increased cognitive function.

In general therefore, there remains a need for alternative and/or more effective compounds that are useful in the treatment and/or prevention of diseases characterised by impaired signalling of neurotrophins and/or other trophic factors, and in particular neurodegenerative diseases such as Alzheimer's disease.

Toltrazuril (1-methyl-3-(3-methyl-4-{4-[(trifluoromethyl)sulfanyl]phenoxy}phenyl)-1,3,5-triazinane-2,4,6-trione; Baycox®) is a triazine-based antiprotozoal compound that is used in veterinary medicine to treat coccidial infections, such as isosporiasis, toxoplasmosis, neosporosis, and equine protozoal meningoencephalitis.

WO2018/115891 discloses that certain 1,3,5-triazine compounds, including toltrazuril and its oxidised variants, are positive modulators of Trk receptor signalling with potential utility in treating diseases characterised by impaired signalling of neurotrophins and/or other trophic factors, such as Alzheimer's disease.

It has now surprisingly been found that certain further 1,3,5-triazine compounds are highly potent modulators of Trk receptor signalling, and therefore have properties rendering them useful for the treatment of diseases characterised by impaired signalling of neurotrophins and/or other trophic factors, such as Alzheimer's disease. These compounds may also display improved metabolic properties for pharmaceutical development.

### DETAILED DESCRIPTION OF THE INVENTION

According to an aspect of the invention, there is provided a compound of formula I

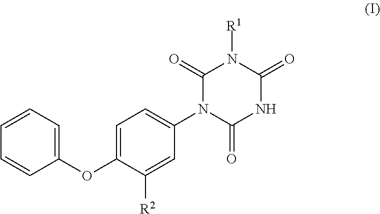

wherein:
R¹ represents or

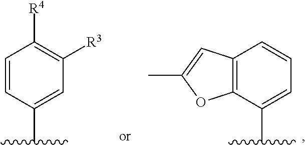

wherein [symbol] represents the point of attachment to the nitrogen atom;
R³ represents hydrogen, methyl or methoxymethyl; and
R⁴ represents hydrogen, methoxy or methoxymethyl;
R² represents methyl, ethyl, methoxymethyl, methylsulfanyl or -A-R⁵; wherein
A represents C₁₋₂ alkylene, —C₁₋₂alkyleneO-, —OC₁₋₂alkylene-, which three groups are optionally substituted by one or more groups selected from halo, C₁₋₂ alkyl and ═O; and
R⁵ represents oxetanyl or a 4-7-membered nitrogen-containing heterocyclyl group, each of which groups is optionally substituted by one or more groups selected from halo, C₁₋₂ alkyl and ═O; and,
or a pharmaceutically-acceptable salt thereof,
with the proviso that the compound of formula I does not represent

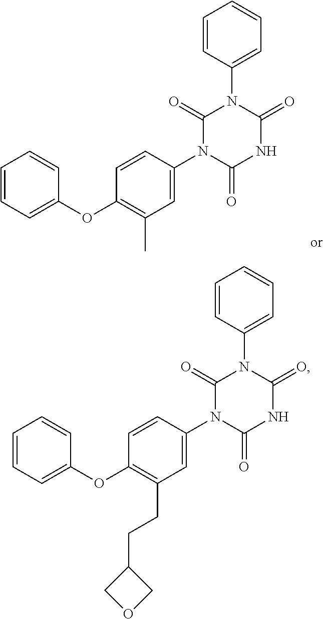 [SMILES: Cc1cc(-n2c(=O)[nH]c(=O)n(-c3ccccc3)c2=O)ccc1Oc1ccccc1 ; O=c1[nH]c(=O)n(-c2ccc(Oc3ccccc3)c(CCC3COC3)c2)c(=O)n1-c1ccccc1]

which compounds (i.e. compounds of formula I, including pharmaceutically-acceptable salts) may be referred to herein as the “compounds of the invention”.

For the avoidance of doubt, the skilled person will understand that references herein to compounds of particular aspects of the invention (such as the first aspect of the invention, i.e. referring to compounds of formula I as defined in the first aspect of the invention) will include references to all embodiments and particular features thereof, which embodiments and particular features may be taken in combination to form further embodiments and features of the invention.

Unless indicated otherwise, all technical and scientific terms used herein will have their common meaning as understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Particular acid addition salts that may be mentioned include those formed by reaction with corresponding acids, thus protonating the compound of the invention, to form carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalene-disulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed by reaction with corresponding bases, thus removing a proton from compounds of the invention, to form salts with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

More particular salts that may be mentioned include Na salts.

For the avoidance of doubt, compounds of the invention may exist as solids, and thus the scope of the invention includes all amorphous, crystalline and part crystalline forms thereof, and may also exist as oils. Where compounds of the invention exist in crystalline and part crystalline forms, such forms may include solvates, which are included in the scope of the invention.

For the avoidance of doubt, compounds of the invention may also exist in solution (i.e. in solution in a suitable solvent). For example, compounds of the invention may exist in aqueous solution, in which case compounds of the invention may exist in the form of hydrates thereof.

Compounds of the invention may also exhibit tautomerism. All tautomeric forms and mixtures thereof are included within the scope of the invention (particularly those of sufficient stability to allow for isolation thereof).

Compounds of the invention may also contain one or more asymmetric carbon atoms and may therefore exhibit optical and/or diastereoisomerism (i.e. existing in enantiomeric or diastereomeric forms). Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired enantiomer or diastereoisomer may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution; for example, with a homochiral acid followed by separation of the diastereomeric derivatives by conventional means such as chromatography), or by reaction with an appropriate chiral reagent or chiral catalyst, all of which methods and processes may be performed under conditions known to the skilled person. Unless otherwise specified, all stereoisomers and mixtures thereof are included within the scope of the invention.

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z} cycloalkyl group). When there is a sufficient number (i.e. a minimum of four) of carbon atoms, such groups may also be part cyclic (so forming a C-z partial cycloalkyl group). For example, cycloalkyl groups that may be mentioned include cyclopropyl, cyclopentyl and cyclohexyl. Similarly, part cyclic alkyl groups (which may also be referred to as “part cycloalkyl” groups) that may be mentioned include cyclopropylmethyl. When there is a sufficient number of carbon atoms, such groups may also be multicyclic (e.g. bicyclic or tricyclic) and/or spirocyclic. For the avoidance of doubt, particular alkyl groups that may be mentioned include straight chain (i.e. not branched and/or cyclic) alkyl groups.

For the avoidance of doubt, alkyl groups as described herein may also act as linker groups (i.e. groups joining two or more parts of the compound as described), in which case such groups may be referred to as “C_{1-z} alkylene” groups. When linker groups are not symmetrical (for example a “—C₁₋₂alkyleneO-” group), it may be understood, in accordance with convention, that the left-hand side of the linker group is attached to the core of the molecule and the right-hand side is attached to the substituent that the group links to the core of the molecule.

As used herein, the term heterocyclyl (group) may refer to non-aromatic monocyclic and polycyclic (e.g. bicyclic) heterocyclic groups (which groups may, where containing a sufficient number of atoms, also be bridged) in which at least one (e.g. one to four) of the atoms in the ring system is other than carbon (i.e. a heteroatom (e.g. nitrogen, oxygen or sulfur)), and in which the total number of atoms in the ring system is between three and twelve (e.g. between five and ten, such as between three and eight; for example, forming a 5- or 6-membered heterocyclyl group). Further, such heterocyclyl groups may be saturated, forming a heterocycloalkyl, or unsaturated containing one or more carbon-carbon or, where possible, carbon-heteroatom or heteroatom-heteroatom double and/or triple bonds, forming for example a C_{2-z} (e.g. C_{4-z}) heterocycloalkenyl (where z is the upper limit of the range) or a C_{7-z} heterocycloalkynyl group.

The term nitrogen-containing heterocyclyl group may be understood to refer to a heterocyclyl group as defined herein, which contains at least one nitrogen atom.

The phrase x-y-membered heterocyclic group, and variations thereof (such as 4-7-membered nitrogen containing heterocyclic group heterocyclic group) may be understood to refer to a heterocyclyl group in which the largest (e.g. the only) ring system contains a total number of atoms that is between x and y.

For the avoidance of doubt, the skilled person will understand that nitrogen-containing heterocyclyl groups that may form part of compounds of the invention are those that are chemically obtainable, as known to those skilled in the art. Various heterocyclyl groups will be well-known to those skilled in the art, such as 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyridinyl, dihydropyrrolyl (including 2,5-dihydropyrrolyl), imidazolidinyl, imidazolinyl, isothiazolidinyl, morpholinyl, 7-oxabicyclo[2.2.1]heptanyl, 6-oxabicyclo[3.2.1]-octanyl, piperazinyl, piperidinyl, pyrazolidinyl, pyrrolidinonyl, pyrrolidinyl, pyrrolinyl, quinuclidinyl, tetrahydropyridinyl (such as 1,2,3,4-tetrahydropyridinyl and 1,2,3,6-tetrahydropyridinyl), thiomorpholinyl, tropanyl and the like.

Substituents on heterocyclyl groups may, where appropriate, be located on any atom in the ring system including a heteroatom. Further, in the case where the substituent is another cyclic compound, then the cyclic compound may be attached through a single atom on the heterocyclyl group, forming a spirocyclic compound. The point of attachment of heterocyclyl groups may be via any suitable atom in the ring system, including (where appropriate) a further heteroatom (such as a nitrogen atom), or an atom on any fused carbocyclic ring that may be present as part of the ring system. Heterocyclyl groups may also be in the N- or S-oxidised forms, as known to those skilled in the art.

For the avoidance of doubt, references to polycyclic (e.g. bicyclic or tricyclic) groups (for example when employed in the context of heterocyclyl or cycloalkyl groups (e.g. heterocyclyl)) will refer to ring systems wherein at least two scissions would be required to convert such rings into a non-cyclic (i.e. straight or branched) chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, to groups in which two non-adjacent atoms are linked by an alkyl (which, when linking two moieties, may be referred to as alkylene) group (optionally containing one or more heteroatoms), which later groups may be referred to as bridged, or to groups in which the second ring is attached to a single atom, which latter groups may be referred to as spiro compounds.

The point of attachment of heterocyclyl groups may be via any atom in the ring system including (where appropriate) a heteroatom. Bicyclic heterocyclyl groups may comprise a non-heterocyclic ring fused, or otherwise connected to one or more further heterocyclic rings, in which instances, the point of attachment of the polycyclic heterocyclyl group may be via any ring including the non-heterocyclic ring.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. compounds of the invention in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more R⁴ groups are present, those R⁴ groups may be the same or different. Similarly, where two or more R⁴ groups are present and each represents C₁₋₂ alkyl optionally substituted by one or more fluoro groups, these groups may also be the same or different.

Further for the avoidance of doubt, when it is specified that a substituent is itself optionally substituted by one or more substituents (e.g. C₁₋₃ alkyl optionally substituted by one or more fluoro groups), these substituents where possible may be positioned on the same or different atoms. Such optional substituents may be present in any suitable number thereof (e.g. the relevant group may be substituted with one or more such substituents, such as one such substituent).

For the avoidance of doubt, where groups are referred to herein as being optionally substituted it is specifically contemplated that such optional substituents may be not present (i.e. references to such optional substituents may be removed), in which case the optionally substituted group may be referred to as being unsubstituted.

For the avoidance of doubt, the skilled person will appreciate that compounds of the invention that are the subject of this invention include those that are obtainable, i.e. those that may be prepared in a stable form. That is, compounds of the invention include those that are sufficiently robust to survive isolation, e.g. from a reaction mixture, to a useful degree of purity.

Compounds of the invention that may be mentioned include those in which R¹ represents

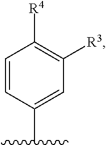

wherein [symbol] , R³ and R⁴ are as defined herein.

Other compound of the invention that may be mentioned include those in which R¹ represents

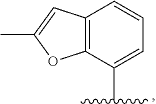

wherein [symbol] represents the point of attachment to the nitrogen atom.

Compounds that may be mentioned include those in which at least one of R³ and R⁴ represents H.

Further compounds of the invention that may be mentioned R³ represents methyl or methoxymethyl and R⁴ represents hydrogen. In particular embodiments, R³ represents methyl and R⁴ represents hydrogen. In further particular embodiments, R³ represents methoxymethyl and R⁴ represents hydrogen.

Further compounds of the invention that may be mentioned include those in which R³ represents hydrogen and R⁴ represents methoxy.

Further compounds of the invention that may be mentioned include those in which R³ and R⁴ both represent hydrogen.

Compounds of the invention that may be mentioned include those in which R² represents methyl, ethyl or methoxymethyl.

Further compounds of the invention that may be mentioned include those in which R² represents methyl.

Further compounds of the invention that may be mentioned include those in which R² represents ethyl or methoxymethyl. In particular embodiments R² represents methoxymethyl.

Other compounds of the invention that may be mentioned include those in which R² represents -A-R⁵, wherein A and R⁵ are as defined herein.

In particular compounds of the invention in which R² represents -A-R⁵, R⁵ represents a 4-7-membered nitrogen-containing heterocyclyl group, which heterocyclyl group is optionally substituted by one or more (e.g. one) groups selected from halo, C₁₋₂ alkyl and, particularly, ═0; more particularly the heterocyclyl group may be substituted by one ═O group or be unsubstituted.

Compounds of the invention that may be mentioned include those in which A represents C₁₋₂ alkylene, —C₁₋₂alkyleneO-, —OC₁₋₂alkylene-, which three groups are optionally substituted by one or more (e.g. one) groups selected from halo (for example chloro or fluoro (e.g. fluoro), C₁₋₂ alkyl (e.g. methyl) and, particularly, ═O.

Further compounds of the invention that may be mentioned include those in which A represents —CH₂— or —OCH₂C(O)—.

Further compounds of the invention that may be mentioned include those in which R⁵ represents a 4-6-membered nitrogen-containing heterocyclyl group, which heterocyclyl group is optionally substituted by one or more groups selected from halo (for example chloro or fluoro (e.g. fluoro), C₁₋₂ alkyl (e.g. methyl) and, particularly, ═O.

Further compounds of the invention that may be mentioned include those in which R⁵ represents oxetanyl (e.g. oxetan-3-yl), in which compounds A preferably represents —CH₂—.

Further compounds of the invention that may be mentioned include those in which R⁵ represents a group selected from:

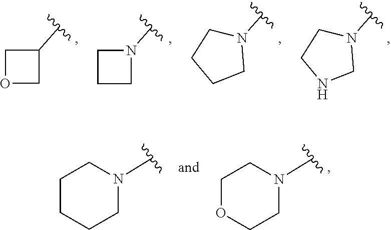

wherein [symbol] represents the point of attachment to A,
each of which heterocyclyl groups is optionally substituted by a ═O group.

Further compounds of the invention that may be mentioned include those in which R⁵ represents a 4-6-membered nitrogen-containing heterocyclyl group selected from:

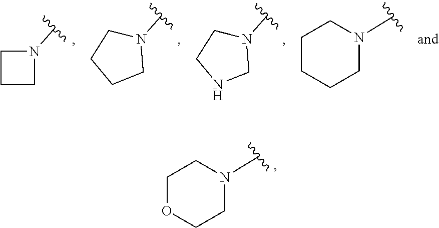

wherein [symbol] represents the point of attachment to A,
each of which heterocyclyl groups is optionally substituted by a ═O group.

Further compounds of the invention that may be mentioned include those in which at least one (e.g. one) of A and R⁵ is substituted by a ═O group.

Further compounds of the invention include those in which -A-R⁵ represents a group selected from

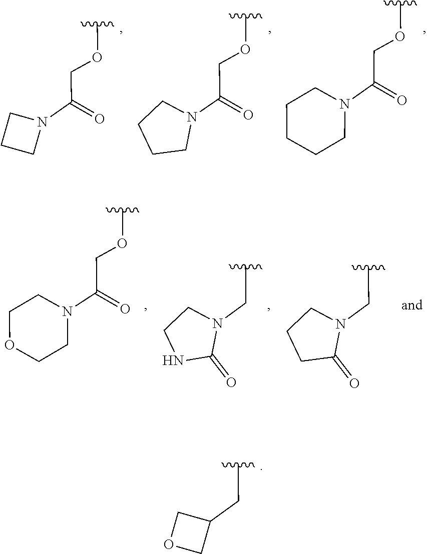

Further compounds of the invention include those in which -A-R⁵ represents a group selected from

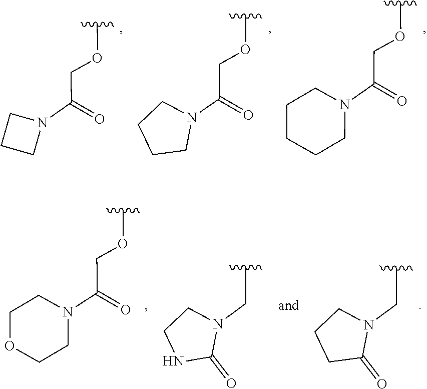

Particular compounds of the invention that may be mentioned include those described in the examples provided herein, and pharmaceutically-acceptable salts thereof. For the avoidance of doubt, where such compounds of the invention include compounds in a particular salt form, compounds of the invention include those compounds in non-salt form and in the form of any pharmaceutically-acceptable salt (which may include the salt form present in the examples).

More particular compounds of the invention include those described in Examples 1, 2, 3, 6 and 7 and pharmaceutically-acceptable salts thereof.

### Medical Uses

As indicated herein, the compounds of the invention, and therefore compositions and kits comprising the same, are useful as pharmaceuticals.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral (e.g. oral) or parenteral administration. All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the active compounds of the invention to which they are metabolised), may also be described as “prodrugs”.

For the avoidance of doubt, compounds of the invention are therefore useful because they possess pharmacological activity, and/or are metabolised in the body following oral or parenteral administration to form compounds that possess pharmacological activity.

As described herein, compounds of the invention may be particularly useful in the treatment of diseases characterised by impaired signalling of neurotrophins and/or other trophic factors. Due to their mode of action, compounds of the invention may have particular utility in the treatment of such diseases in patients with the Val66Met mutation in the BDNF gene.

The compounds of the invention may also have particular utility in the treatment of diseases characterised by impaired signalling of neurotrophins and/or other trophic factors in patients having other genetic variations, including deletions, that directly or indirectly affect the BDNF gene. For example, the compounds of the invention may have particular utility in treating diseases in patients having the rs12291063 minor C allele, which is known to be associated with lower BDNF expression, and/or in patients having the deletions associated with WAGR syndrome, such as the deletions in chromosome 11.

Accordingly, in particular embodiments of the invention, there is provided the compounds of the invention for use in the treatment of the diseases described herein in a patient having the Val66Met mutation in the BDNF gene, and/or in a patient having the rs12291063 minor C allele, and/or in a patient having the genetic deletions associated with WAGR syndrome.

The skilled person will understand that trophic factors refer to a class of molecules that promote the growth and maintenance of cellular tissues. Neurotrophins may be understood to refer to a class of molecules associated with promoting the growth and survival of neurons, which are also referred to as neurotrophic factors. Examples of neurotrophins include NGF, BDNF, NT3 and NT4/5. Other trophic factors include insulin-like growth factor (IGF-1), fibroblast growth factors (FGFs), hepatocyte growth factor (HGF) and glial cell line-derived neurotrophic factors such as glial cell-derived neurotrophic factor (GDNF), Neurturin (NRTN), artemin (ARTN) and persephin (PSPN).

As used herein, the phrase diseases characterised by impaired signalling of neurotrophins and other trophic factors may be understood to indicate diseases and disorders that involve reduced signalling of trophic factors, such as those listed above. Such disorders may be treated through the positive modulation of neurotrophin receptors, such as TrKA, TrKB and TrkC and/or their signalling, and receptor tyrosine kinases such as FGFR1 and IGF1R and/or their signalling and/or the positive modulation of other trophic factor receptors.

The Val66Met mutation in the BDNF gene refers to a common single-nucleotide polymorphism in the brain-derived neurotrophic factor (BDNF) gene, resulting in a methionine (Met) substitution for valine (Val) at codon 66 (Val66Met).

The skilled person will understand that references to the treatment of a particular condition (or, similarly, to treating that condition) will take their normal meanings in the field of medicine. In particular, the terms may refer to achieving a reduction in the severity and/or frequency of occurrence of one or more clinical symptom associated with the condition, as adjudged by a physician attending a patient having or being susceptible to such symptoms. For example, in the case of Alzheimer's disease, the term may refer to achieving an improvement in cognition in the patient being treated.

As used herein, the term prevention (and, similarly, preventing) will include references to the prophylaxis of the disease or disorder (and vice versa). As such, references to prevention may also be references to prophylaxis, and vice versa. In particular, such terms may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that patient is diagnosed by a physician as having, e.g. requiring treatment for, the relevant disease or disorder).

As used herein, references to a patient (or to patients) will refer to a living subject being treated, including mammalian (e.g. human) patients.

For the avoidance of doubt, the skilled person will understand that such treatment or prevention will be performed in a patient (or subject) in need thereof. The need of a patient (or subject) for such treatment or prevention may be assessed by those skilled the art using routine techniques.

As used herein, the terms disease and disorder (and, similarly, the terms condition, illness, medical problem, and the like) may be used interchangeably.

Compounds of the invention are modulators of neurotrophin receptors, such as TrkA, TrkB, TrkC and/or their signalling and receptor tyrosine kinases, such as FGFR1 and IGF1R and/or their signalling. The compounds are believed to have an improved potency for the modulation of neurotrophin receptors, such as TrkA, TrkB, TrkC and/or their signalling and receptor tyrosine kinases, such as FGFR1 and IGF1R and/or their signalling. It is believed that the compounds of the invention would have a reduced potential for side effects associated with conventional agonists for TrkA and TrkB.

Another indication includes setting in which there is a goal for enhancing plasticity of the nervous system, such as during rehabilitation or acquisition of a new learned physical or intellectual skill. Moreover, it also includes facilitation of neuronal or non-neuronal or stem cell survival or promoting neural function by treating a neural or non-neuronal or stem cell with a compound of the invention having the ability to have a positive modulatory effect, either directly or indirectly, on the signalling mediated by the TrkA, TrkB and TrkC receptors, optionally in combination with a modulatory effect, either directly or indirectly, on the signalling mediated by receptor tyrosine kinases such as IGF1R and/or FGFR1 receptor.

The invention relates to the compounds of the invention and pharmaceutically-acceptable salts thereof, as defined above, for use in medicine (e.g. human medicine). Without being bound to theory regarding the mode of action of the compounds defined above, it is believed that the compounds can be used for treatment and/or prevention of the diseases mentioned herein.

In particular embodiments, the diseases that may be treated by compounds of the invention include Alzheimer's disease, Lewy body dementia, frontotemporal dementia, cognitive dysfunction, mild cognitive impairment, other dementia disorders, Parkinson's disease, other Parkinsonian disorders and/or other tauopathies, Huntington's disease, brain injuries (including traumatic brain injuries), stroke, motor neurone diseases, disorders in which enhancement of nerve regeneration is beneficial (such as demyelinating diseases including multiple sclerosis), spinal chord injury, hypoxia, ischemia, hypoxic ischemia injury, coronary artery disease, obesity, diabetes, metabolic syndrome, diabetes, diabetic neuropathy (including complications thereof such as osteoporosis (diabetes-induced osteoporosis), painful connective tissue disorders and tendon ruptures), Charcot-Marie-tooth disease and its variants, nerve injury (including peripheral nerve injury), hearing loss (including genetic, acquired or traumatic hearing loss), blindness, posterior eye diseases, anterior eye diseases, dry eye syndrome, neurotrophic keratitis, glaucoma, high intraocular pressure (IOP), retinitis pigmentosa, post-traumatic stress disorders, WAGR syndrome, Prader-Willi syndrome, diseases of the olfactory tract, olfactory decline, olfactory dysfunction, fragile X syndrome, congenital central hypoventilation syndrome, obsessive-compulsive disorder, anxiety, generalized anxiety disorder, schizophrenia, depression, eating disorders, bipolar disorder, chronic fatigue syndrome, neuromyelitis optica, Rett syndrome, epilepsy, Friedreich's ataxia, obstructive sleep apnea-hypopnea syndrome, pain and constipation (including, particularly, constipation in Parkinson's disease, slow-transit constipation and opioid-induced constipation).

As used herein, the phrase “other Parkinsonian disorders” may be understood to refer to disorders that have symptoms similar to Parkinson's disease, such as bradykinesia, tremors and postural instability. Examples of such disorders include progressive supranuclear palsy (PSP), multiple system atrophy (MSA), and corticobasal degeneration (CBD).

The phrase “other tauopathies” may be understood to refer to neurodegenerative diseases other than Alzheimer's disease that are associated with the pathological misfolding of tau protein in the brain. Examples of such disorders include primary age-related tauopathy, progressive supranuclear palsy, Pick's disease, corticobasal degeneration and post-encephalitic parkinsonism. The skilled person will understand that certain disorders such as progressive supranuclear palsy may be described as both a Parkinsonian disorder and a tauopathy.

The phrase “other dementia disorders” may be understood to include vascular dementia, mixed vascular dementia, incident dementia, post-operative dementia, presenile dementia, dementia associated with Parkinson's disease and dementia due to HIV infection. Progressive supranuclear palsy and corticobasal degeneration may also be classed as dementia disorders.

Motor neurone diseases include amyotrophic lateral sclerosis (ALS), hereditary spastic paraplegia (HSP), primary lateral sclerosis (PLS), progressive muscular atrophy (PMA), progressive bulbar palsy (PBP) and pseudobulbar palsy.

Cognitive dysfunction may be understood to refer to reduced cognitive abilities in a patient including reduced ability in learning, memory loss, perception, and problem solving. Cognitive dysfunction is associated with a range of conditions, such as Alzheimer's disease, Parkinson's disease, progressive supranuclear palsy, corticobasal degeneration and schizophrenia. Accordingly, in particular embodiments, the compounds of the invention may be used in the treatment of cognitive dysfunction in Alzheimer's disease, Parkinson's disease, progressive supranuclear palsy, corticobasal degeneration or schizophrenia. Cognitive dysfunction also includes post-operative cognitive dysfunction and impaired cognition associated with preterm delivery.

Similarly, in other particular embodiments, the compounds of the invention may be used in improving cognition in a patient with Alzheimer's disease, Parkinson's disease, progressive supranuclear palsy, corticobasal degeneration or schizophrenia. As used herein, the phrase “improving cognition” may be understood to indicate enhancing a patient's learning, memory, perception, and/or problem-solving ability. Improving cognition may also refer to slowing or arresting the rate of decline in cognition in a patient suffering from cognitive dysfunction (e.g. associated with the disorders listed above).

Cognitive function may be assessed using standard tests known to the person skilled in the art. Examples of such tests include the Alzheimer's Disease Assessment Scale-Cognitive subscale test (ADAS-COG) the Mini-Mental State Examination (MMSE), the Clinical Dementia Rating (CDR) the Clinical Dementia Rating-Sum of Boxes (CDR-SB), the Alzheimer's Disease Cooperative Study—Preclinical Alzheimer Cognitive Composite (ADCS-PACC) and the Repeatable Battery for the Assessment of Neuropsychological Status (RBANS) test.

As used herein, “eating disorders” may be understood to include hyperphagia, anorexia nervosa, restricting anorexia nervosa and bulimia nervosa.

In a further embodiment of the invention, the compounds of the invention may for use in improving tissue regeneration, improving motor function, nerve transplantation and treating complications associated with nerve transplantation.

In accordance with a further aspect of the invention, there is provided the compounds of the invention, or a pharmaceutically acceptable salt thereof, for use in treatment and/or prevention of one or more disease selected from the group comprising or containing Alzheimer's disease, Lewy body dementia, frontotemporal dementia, HIV dementia, Huntington's disease, amyotrophic lateral sclerosis and other motor neurone diseases, Rett syndrome, epilepsy, Parkinson's disease and other parkinsonian disorders, disorders in which enhancement of nerve regeneration is beneficial, such as demyelinating diseases including multiple sclerosis, spinal cord injury, stroke, hypoxia, ischemia, brain injury including traumatic brain injury, mild cognitive impairment, dementia disorders (including dementia of mixed vascular and degenerative origin, presenile dementia, senile dementia and dementia associated with Parkinson's disease, progressive supranuclear palsy or corticobasal degeneration) and cognitive dysfunction in schizophrenia, obesity, diabetes and metabolic syndrome, diabetic neuropathy including associated disorders such as osteoporosis, painful connective tissue disorders and tendon ruptures, Charcot Marie Tooth disease and its variants, nerve transplantation and its complications, motor neurone disease, peripheral nerve injury, genetic, acquired or traumatic hearing loss, blindness and posterior eye diseases, glaucoma, dry eye syndrome, neurotrophic keratitis, depression, obesity, metabolic syndrome, pain, depression, schizophrenia and anxiety.

In more particular embodiments, the disease characterised by impaired signalling of neurotrophins and/or other trophic factors is selected from the group consisting of Alzheimer's disease, Parkinson's disease, other Parkinsonian diseases, other tauopathies, Lewy body dementia, motor neurone disease, Pick's disease, obesity, metabolic syndrome, diabetes, diabetic neuropathy, glaucoma, dry eye syndrome, neurotrophic keratitis, genetic, acquired or traumatic hearing loss and Rett syndrome.

In more particular embodiments, the disease characterised by impaired signalling of neurotrophins and/or other trophic factors is selected from the group consisting of Alzheimer's disease, Parkinson's disease, other Parkinsonian diseases, other tauopathies, Lewy body dementia, motor neurone disease, Pick disease, obesity, metabolic syndrome, diabetes, glaucoma, dry eye syndrome, neurotrophic keratitis, genetic, acquired or traumatic hearing loss and Rett syndrome. The treatment of this group of disorders may be particularly effective in patients having the Val66Met mutation in the BDNF gene.

In yet more particular embodiments, the disease characterised by impaired signalling of neurotrophins and/or other trophic factors is an eye disorder.

In yet more particular embodiments, the disease characterised by impaired signalling of neurotrophins and/or other trophic factors is an eye disorder selected from the group consisting of blindness, posterior eye diseases, anterior eye diseases, dry eye syndrome, neurotrophic keratitis, glaucoma, high intraocular pressure and retinitis pigmentosa. More particularly, the eye disorder is selected from the group consisting of dry eye syndrome, neurotrophic keratitis and glaucoma.

In yet more particular embodiments, the disease characterised by impaired signalling of neurotrophins and/or other trophic factors is selected from the group consisting of Alzheimer's disease, Parkinson's disease, Cognitive dysfunction, depression, diabetic neuropathy, glaucoma, dry eye syndrome, neurotrophic keratitis, genetic, acquired or traumatic hearing loss and Rett Syndrome.

In yet more particular embodiments, the disease characterised by impaired signalling of neurotrophins and/or other trophic factors is selected from the group consisting of Alzheimer's disease, Parkinson's disease, Cognitive dysfunction, depression and Rett Syndrome.

A further embodiment of the invention relates to a compound of the invention, or a pharmaceutically acceptable salt thereof, for use in treatment and/or prevention of Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and/or other Parkinsonian disorders.

A further embodiment of the invention relates to a compound of the invention, or a pharmaceutically acceptable salt thereof, for use in treatment and/or prevention of Alzheimer's disease, Parkinson's disease, Cognitive dysfunction in Schizophrenia, Rett's Syndrome and/or depression.

A further embodiment of the invention relates to a compound of the invention, or a pharmaceutically acceptable salt thereof, for use in treatment and/or prevention of Alzheimer's disease.

A further embodiment of the invention relates to a compound of the invention, or a pharmaceutically acceptable salt thereof, for use in treatment and/or prevention of depression.

A further embodiment of the invention relates to a compound of the invention, or a pharmaceutically acceptable salt thereof, for use in treatment and/or prevention of a disease where enhancement of nerve regeneration is beneficial, such as demyelinating diseases.

A further embodiment of the invention relates to a compound of the invention, or a pharmaceutically acceptable salt thereof, for use in treatment and/or prevention of multiple sclerosis.

A further embodiment of the invention relates to a compound of the invention, or a pharmaceutically acceptable salt thereof, for use in treatment and/or prevention of Rett syndrome.

A further embodiment of the invention relates to a compound of the invention, or a pharmaceutically acceptable salt thereof, for use in treatment and/or prevention spinal cord injury, stroke, hypoxia, ischemia and/or brain injury including traumatic brain injury.

A further embodiment of the invention relates to a compound of the invention, or a pharmaceutically acceptable salt thereof, for use in the treatment and/or prevention of mild cognitive impairment, dementia disorders (including dementia of mixed vascular and degenerative origin, presenile dementia, senile dementia and dementia associated with Parkinson's disease, progressive supranuclear palsy, corticobasal degeneration, post-operative dementia) and/or cognitive dysfunction in schizophrenia.

A further embodiment of the invention relates to a compound of the invention, or a pharmaceutically acceptable salt thereof, for use in the treatment and/or prevention of atherosclerosis, obesity, diabetes and metabolic syndrome, diabetic neuropathy including complications thereof such as osteoporosis, painful connective tissue disorders and tendon ruptures, Charcot Marie Tooth disease and its variants, nerve transplantation and its complications, diabetes induced osteoporosis, motor neurone disease, peripheral nerve injury, genetic or acquired or traumatic hearing loss, blindness and posterior eye diseases, depression, obesity, metabolic syndrome, WAGR syndrome, Prader Willi syndrome and/or pain.

A further embodiment of the invention relates to a compound of the invention, or a pharmaceutically acceptable salt thereof, for use in the treatment and/or prevention of atherosclerosis, obesity, diabetes and metabolic syndrome, diabetic neuropathy including complications thereof such as osteoporosis, painful connective tissue disorders and tendon ruptures, Charcot Marie Tooth disease and its variants, nerve transplantation and its complications, motor neurone disease, peripheral nerve injury, genetic or acquired or traumatic hearing loss, blindness and posterior eye diseases, depression, obesity, metabolic syndrome and/or pain.

A further embodiment of the invention relates to a compound of the invention, or a pharmaceutically acceptable salt thereof, for use in the treatment and/or prevention of depression, schizophrenia and/or anxiety.

Another embodiment relates to a use of a compound of the invention, or a pharmaceutically acceptable salt thereof, for the treatment and/or prevention of a disease in which modulators of neurotrophin receptors, such as TrkA, TrkB, TrkC and/or their signalling and receptor tyrosine kinases, such as FGFR1 and IGF1R and/or their signalling are beneficial, such as for the treatment and/or prevention of both non-neurological and neurological diseases, including one or more of the conditions mentioned hereinbefore.

The invention further relates to the use of a compound of the invention in a method of treating, preventing or reducing the risk of a disease in which modulators of neurotrophin receptors, such as TrkA, TrkB, TrkC and/or their signalling and receptor tyrosine kinases, such as FGFR1 and IGF1R and/or their signalling, are beneficial, such as in the treatment and/or prevention of both non-neurological and neurological diseases.

One embodiment relates to the use of a compound of the invention (for example in the manufacture of a pharmaceutical medicament) for use in a method of treating, preventing or reducing the risk of, one or more disease mentioned hereinbefore, which comprises administering to a mammal, such as a human, in need thereof, a therapeutically effective amount of a compound of the invention, or a pharmaceutically acceptable salt thereof.

Another embodiment relates to such a use of a compound of the invention in a method of treating, preventing or reducing the risk of Alzheimer's disease, Lewy body dementia, frontotemporal dementia, HIV dementia, Huntington's disease, amyotrophic lateral sclerosis and other motor neuron diseases, Rett syndrome, epilepsy, Parkinson's disease and/or other parkinsonian disorders.

A further embodiment relates to such a use of a compound of the invention in a method of treating, preventing or reducing the risk of Alzheimer's disease, Parkinson's disease, Cognitive dysfunction in Schizophrenia, Rett's Syndrome and/or Depression.

A further embodiment relates to such a use of a compound of the invention in a method of treating, preventing or reducing the risk of a disease where enhancement of nerve regeneration is beneficial such as demyelinating diseases, such as multiple sclerosis.

A further embodiment relates to such a use of a compound of the invention in a method of treating, preventing or reducing the risk of spinal cord injury, stroke, hypoxia, ischemia and/or brain injury including traumatic brain injury.

A further embodiment relates to such a use of a compound of the invention in a method of treating or preventing constipation, particularly constipation in Parkinson's disease, slow-transit constipation and opioid-induced constipation.

Another embodiment relates to such a use of a compound of the invention in a method of treating, preventing or reducing the risk of mild cognitive impairment, dementia disorders (including dementia of mixed vascular and degenerative origin, presenile dementia, senile dementia and dementia associated with Parkinson's disease, progressive supranuclear palsy or corticobasal degeneration) and/or cognitive dysfunction in schizophrenia.

A further embodiment relates to such a use of a compound of the invention in a method of treating, obesity, diabetes and metabolic syndrome, diabetic neuropathy including complications thereof such as osteoporosis, painful connective tissue disorders and tendon ruptures, Charcot Marie Tooth disease and its variants, nerve transplantation and its complications, diabetes-induced osteoporosis, motor neuron disease, peripheral nerve injury, genetic or acquired or traumatic hearing loss, blindness and posterior eye diseases, depression, obesity, metabolic syndrome and/or pain.

Yet another embodiment relates to such a use of a compound of the invention in a method of treating, preventing or reducing the risk of depression, schizophrenia and/or anxiety.

As described above, treatment of the disorders described herein with the compounds of the invention may be particularly effective in patients with the Val66Met mutation in the BDNF gene. Accordingly, in particular embodiments, the treatment of the disorders characterised by impaired signalling of neurotrophins and/or other trophic factors as defined herein (including the various embodiments described herein) is in a patient with the Val66Met mutation in the BDNF gene.

### Pharmaceutical Compositions

As described herein, compounds of the invention are useful as pharmaceuticals. Such compounds may be administered alone or may be administered by way of known pharmaceutical compositions/formulations.

In a further aspect of the invention, there is provided a pharmaceutical composition comprising a compound of the invention, or a pharmaceutically-acceptable salt thereof, and a pharmaceutically-acceptable excipient, such as a pharmaceutically-acceptable adjuvant, diluent or carrier, for use in the treatment of a disease characterised by impaired signalling of neurotrophins and/or other trophic factors (including the various diseases and disorders listed herein), optionally in a patient with the Val66Met mutation in the BDNF gene.

As used herein, the term pharmaceutically-acceptable excipients includes references to vehicles, adjuvants, carriers, diluents, pH adjusting and buffering agents, tonicity adjusting agents, stabilizers, wetting agents and the like. In particular, such excipients may include adjuvants, diluents or carriers.

The skilled person will understand that compounds of the invention may act systemically and/or locally (i.e. at a particular site), and may therefore be administered accordingly using suitable techniques known to those skilled in the art. In particular, the compounds may be administered to act systemically, for example through oral administration.

The skilled person will understand that compounds and compositions as described herein will normally be administered orally, intravenously, subcutaneously, buccally, rectally, dermally, nasally, tracheally, bronchially, sublingually, intranasally, topically (including topical administration to the eyes), by any other parenteral route or via inhalation, in a pharmaceutically acceptable dosage form.

Conventional procedures for the selection and preparation of suitable pharmaceutical formulations are described in, for example, “Pharmaceuticals—The Science of Dosage Form Designs”, M. E. Aulton, Churchill Livingstone, 1988. For preparing pharmaceutical compositions from the compounds of the invention, inert, pharmaceutically acceptable carriers can be either solid or liquid. Solid form preparations include powders, tablets, dispersible granules, capsules, cachets, and suppositories.

Pharmaceutical compositions as described herein will include formulations in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration. In particular, compounds may be formulated for local delivery to the CNS, for example in the form of artificial cerebrospinal fluid (CSF).

Thus, in particular embodiments, the pharmaceutical composition is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

Thus, compounds, of the present invention, and compositions comprising the same, may be administered orally, parenterally, buccally, vaginally, rectally, by inhalation, by insufflation, sublingually, intramuscularly, subcutaneously, topically (including topical administration to the eyes), intranasally, intraperitoneally, intrathoracically, intravenously, epidurally, intrathecally, intracerebroventricularly and by injection into the joints.

Depending on the mode of administration, pharmaceutical compositions will preferably comprise from 0.05 to 99% wt (percent by weight), more preferably from 0.05 to 80% wt, still more preferably from 0.10 to 70% wt, and even more preferably from 0.10 to 50% wt, of a compounds of the invention (calculated as a non-salt form), all percentages by weight being based on total composition.

Depending on e.g. potency and physical characteristics of the compound of the invention (i.e. active ingredient), pharmaceutical formulations that may be mentioned include those in which the active ingredient is present in an amount that is at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The quantity of the compound to be administered will vary for the patient being treated and will vary from about 100 ng/kg of body weight to 100 mg/kg of body weight per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in uses or methods of the invention.

More, particularly, the skilled person will understand that compounds of the invention may be administered (for example, as formulations as described hereinbefore) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 14 mg/kg/day, preferably about 0.01 μg/kg/day to about 10 mg/kg/day, and more preferably about 0.1 μg/kg/day to about 5.0 mg/kg/day. For example, when administered orally, treatment with such compounds may comprise administration of a formulations typically containing between about 0.01 μg to about 1000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 1 mg, 5 mg, 10 mg, 25 mg, 50 mg, 100 mg or 200 mg twice daily).

For the avoidance of doubt, the skilled person (e.g. the physician) will be able to determine the actual dosage which will be most suitable for an individual patient, which is likely to vary with the route of administration, the type of formulation, the type and severity of the condition that is to be treated, other medication the patient may be taking, as well as the species, age, weight, size, sex, diet, renal function, hepatic function, general physical condition, genetic factors and response of the particular patient to be treated. Although the above-mentioned dosages are exemplary of the average case, there can, of course, be individual instances where higher or lower dosage ranges are merited, and such doses are within the scope of the invention.

Thus, in a further aspect of the invention, there is provided a use of a pharmaceutical composition, as defined above, in therapy, or for the treatment and/or prevention of a disease characterised by impaired signalling of neurotrophins and/or other trophic factors.

### Combinations and Kits-of-Parts

The treatment and/or prevention of diseases of the nervous system and related pathologies defined herein may comprise administration of a compound of the invention as a sole therapy or may involve, in addition to the compound of the invention, conjoint treatment with conventional therapy of value in treating one or more disease conditions referred to herein. Such conventional therapy may include one or more agents such as acetyl cholinesterase inhibitors, anti-inflammatory agents, cognitive and/or memory enhancing agents, atypical antipsychotic agents, dopamine agonists and/or L-DOPA.

Such conjoint treatment and/or prevention may be achieved by way of the simultaneous, sequential or separate dosing of the individual compounds of the invention or additional agents of the treatment and/or prevention. Such combination products employ the compounds, or pharmaceutically-acceptable salts thereof, of the invention.

Accordingly, the skilled person will understand that treatment with compounds of the invention may further comprise (i.e. be combined with) further treatment(s) or preventative methods for the same condition. In particular, treatment with compounds of the invention may be combined with means for the treatment of diseases characterised by impaired signalling of neurotrophins and/or other trophic factors (such as Alzheimer's disease, Parkinson's disease, cognitive dysfunction and depression as described herein, e.g. Alzheimer's disease) such as treatment with one or more other therapeutic agent that is useful in the in the treatment the various diseases characterised by impaired signalling of neurotrophins and/or other trophic factors described herein, and/or one or more physical method used in the treatment (such as treatment through surgery), as known to those skilled in the art.

As described herein, compounds of the invention may also be combined with one or more other (i.e. different) therapeutic agents (i.e. agents that are not compounds of the invention) that are useful in the treatment and/or prevention of diseases characterised by impaired signalling of neurotrophins and/or other trophic factors. Such combination products that provide for the administration of a compound of the invention in conjunction with one or more other therapeutic agent may be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the one or more other therapeutic agent).

Thus, according to a further aspect of the invention, there is provided a combination product comprising:
(I) a compound of the invention as hereinbefore defined, or a pharmaceutically acceptable salt thereof; and
(II) one or more other therapeutic agent that is useful in the treatment or prevention of a disease characterised by impaired signalling of neurotrophins and/or other trophic factors,
wherein each of components (I) and (II) is formulated in admixture, optionally with a pharmaceutically-acceptable excipient, such as a pharmaceutically-acceptable adjuvant diluent or carrier.

According to a further aspect of the invention there is provided a pharmaceutical composition comprising a compound of the invention as hereinbefore defined, or a pharmaceutically acceptable salt thereof and one or more other therapeutic agent that is useful in the treatment or prevention of a disease characterised by impaired signalling of neurotrophins and/or other trophic factors, formulated together in admixture, optionally with a pharmaceutically-acceptable excipient, such as a pharmaceutically-acceptable adjuvant diluent or carrier.

According to a further aspect of the invention, there is provided a kit-of-parts comprising:
(a) a pharmaceutical composition comprising a compound of the invention as hereinbefore defined, or a pharmaceutically acceptable salt thereof, formulated in admixture, optionally with a pharmaceutically-acceptable excipient, such as a pharmaceutically-acceptable adjuvant diluent or carrier; and
(b) a pharmaceutical composition comprising one or more other therapeutic agent that is useful in the treatment or prevention of a disease characterised by impaired signalling of neurotrophins and/or other trophic factors, formulated in admixture, optionally with a pharmaceutically-acceptable excipient, such as a pharmaceutically-acceptable adjuvant diluent or carrier,
which components (a) and (b) are each provided in a form that is suitable for administration in conjunction with the other.

With respect to the kits-of-parts as described herein, by “administration in conjunction with” (and similarly “administered in conjunction with”) we include that respective formulations are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients are administered (optionally repeatedly) either together, or sufficiently closely in time, to enable a beneficial effect for the patient, that is greater, over the course of the treatment and/or prevention of the relevant condition, than if either agent is administered (optionally repeatedly) alone, in the absence of the other component, over the same course of treatment and/or prevention. Determination of whether a combination provides a greater beneficial effect in respect of, and over the course of, treatment or prevention of a particular condition will depend upon the condition to be treated or prevented, but may be achieved routinely by the skilled person.

Further, in the context of the present invention, the term “in conjunction with” includes that one or other of the two formulations may be administered (optionally repeatedly) prior to, after, and/or at the same time as, administration of the other component. When used in this context, the terms “administered simultaneously” and “administered at the same time as” includes instances where the individual doses of the compound of the invention and the additional compound for the treatment of a disease characterised by impaired signalling of neurotrophins and/or other trophic factors, or pharmaceutically acceptable salts thereof, are administered within 48 hours (e.g. within 24 hours, 12 hours, 6 hours, 3 hours, 2 hours, 1 hour, 45 minutes, 30 minutes, 20 minutes or 10 minutes) of each other.

Other therapeutic agents useful in the treatment or prevention of diseases characterised by impaired signalling of neurotrophins and/or other trophic factors will be well-known to those skilled in the art. For example, such other therapeutic agents may include: acetyl cholinesterase inhibitors, anti-inflammatory agents, cognitive enhancing agents, memory enhancing agents, and atypical antipsychotic agents, anti-depressive agents, anti-Alzheimer's agents, beta-secretase inhibitors, gamma-secretase modulators, agents modifying tau function, amyloid-beta production inhibitors, antibodies directed at amyloid-beta, antibodies directed at tau, antibodies directed at alpha-synuclein, anti-Parkinson agents, anti-diabetic agents, anti-multiple sclerosis agents, anti-obesity agents, agents used for treatment of auditory dysfunction, agents used for treatment of ocular disease, agents used for the treatment of olfactory dysfunction, agents used for the treatment of gustatory dysfunction, anti-Huntington agents, anti-Rett syndrome agents, anti-stroke agents. Particular therapeutic agents that may be mentioned include acetyl cholinesterase inhibitors, anti-Alzheimer's agents, anti-Parkinson agents, cognitive enhancing agents, antibodies directed at amyloid-beta, antibodies directed at tau, antibodies directed at alpha-synuclein, beta-secretase inhibitors and gamma-secretase modulators, anti-constipation agents (such as laxatives, serotonin agonists and chloride channel activators).

### Preparation of Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable excipient.

In further aspects of the invention, there is provided a process for the preparation of a combination product or kit-of-parts as hereinbefore defined, which process comprises bringing into association a compound of the invention, as hereinbefore defined, with the other therapeutic agent that is useful in the treatment of the relevant disease or disorder, and at least one pharmaceutically-acceptable excipient.

As used herein, references to bringing into association will mean that the two components are rendered suitable for administration in conjunction with each other.

Thus, in relation to the process for the preparation of a kit-of-parts as hereinbefore defined, by bringing the two components “into association with” each other, we include that the two components of the kit-of-parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

### Preparation of Compounds of the Invention

Compounds of the invention as described herein may be prepared either as a free base or as a pharmaceutically acceptable salt in accordance with techniques that are well known to those skilled in the art, such as those described in the examples provided hereinafter.

According to a further aspect of the invention there is provided a process for the preparation of a compound of the invention, which comprises the step of reacting a compound of formula II

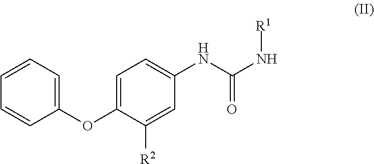

wherein R¹ and R² are as defined herein with a suitable isocyanate (e.g. methoxycarbonyl isocyanate, chlorocarbonyl isocyanate or, preferably, ethoxycarbonyl isocyanate) in the presence of a suitable solvent (e.g. chlorobenzene or bromobenzene), and at a suitable reaction temperature (in particular the reaction may be performed at a temperature of greater than 100° C. (e.g. 150° C.) in a suitable sealed vessel, optionally undermicrowave irradiation).

Compounds of formula II may be obtained by reacting a compound of formula III

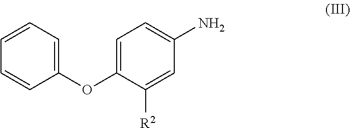

wherein R² is as defined herein, with either:
(a) a compound of formula IV,

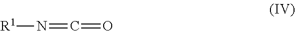

in the presence of a suitable solvent (e.g. DMF) and at a suitable temperature (e.g. between 0° C. and room temperature); or
(b) a compound of formula V,

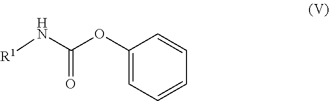

wherein R¹ is as defined herein, in the presence of a suitable solvent (e.g. DMF) and a suitable base (e.g. triethylamine) at a suitable temperature (e.g. between room temperature and reflux temperature); or
(b) a compound of formula VI

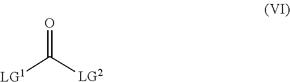

wherein LG¹ and LG² represent suitable leaving groups independently selected from chloro, methoxy, ethoxy and 1-imidazolyl (particular compounds of formula X that may be mentioned include triphosgene and carbonyl diimidazole), in a suitable solvent (e.g. dichloromethane) at a suitable temperature (e.g. between 0° C. and room temperature) for a suitable period of time (for example between one and six hours), and after which time adding a compound of formula VII

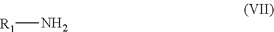

wherein R¹ is as defined herein, and optionally a suitable base (e.g. NaHCO₃ or triethylamine).

Step (b) may alternatively be performed by reacting the compound of VII with a compound of formula (VI) followed by a compound of formula III, under substantially the same reaction conditions as defined above.

Compounds of formula III may be obtained by reducing a compound of formula (VII)

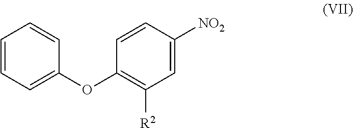

wherein R² is as defined herein, in the presence of a suitable reducing agent such as SNCl₂.2H₂O, for example in the presence of HCl, or using Pd/C in the presence of H₂(g). This reaction may be performed in a suitable, such as ethanol, and at a suitable temperature (for example between room temperature and reflux temperature).

In a further embodiment of the invention, there is provided a process for the preparation of a compound of the invention, which comprises the step of reacting a compound of formula VIII,

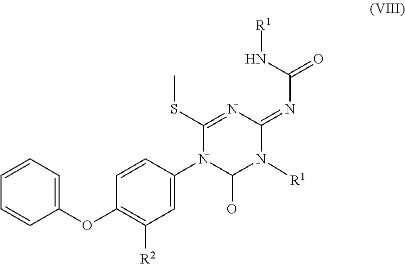

wherein R¹ and R² are as defined herein, in the presence of an aqueous acid (such as HCl (e.g. 2M HCl)) and optionally an organic co-solvent (e.g. 1,4-dioxane), and at a suitable temperature (e.g. between room temperature and reflux temperature).

Compounds of formula VIII may be obtained by reacting a compound of formula IX,

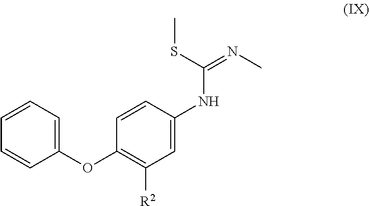

wherein R² is as defined herein, with an excess (e.g. 1.5 or 2 equivalents) of a compound of formula IV as defined herein and a compound of formula VI as defined herein in the presence of a suitable base (e.g triethylamine) and a suitable solvent (e.g. dichloromethane or acetonitrile), at a suitable temperature (e.g. room temperature to reflux temperature).

Compounds of formula VIII may be obtained by reacting a compound of formula III as defined herein with dimethyl dimethyl N-cyanodithioiminocarbonate in the presence of a suitable solvent (e.g. ethanol) at a suitable temperature (e.g. reflux temperature) for a suitable period of time (e.g. 24 hours to 10 days).

Certain compounds of the invention in which R² represents -A-R⁵ and R⁵ represents a nitrogen-linked 4-7-membered nitrogen-containing heterocyclyl group may also be obtained by reacting a compound of formula X,

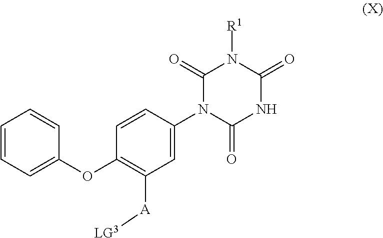

wherein R¹ is as defined herein and LG³ represents a suitable leaving group (e.g. bromo or, particularly, chloro), with a compound of formula X,

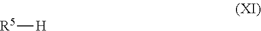

wherein R⁵ is as defined herein provided that H (as drawn in the compound of formula XI) is attached to a nitrogen atom, in the presence of a suitable base (e.g. NaH) and a suitable solvent (e.g. THF) at a suitable temperature (e.g. between 0° C. and room temperature).

Certain compounds of the invention in which R² represents -A-R⁵ may also be obtained by reacting a compound of formula XII,

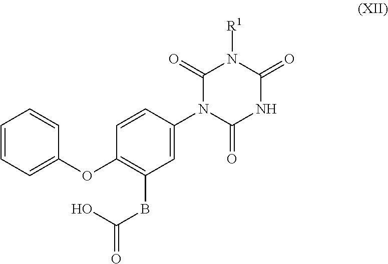

wherein R¹ is as defined herein and B represents —CH₂— or, particularly, —OCH₂—, with a compound of formula XI as defined herein, in the presence of a suitable coupling agent (i.e. an agent suitable for activating the carboxylic acid moiety and thereby facilitating amide bond formation, such as a carbodiimide coupling agent (e.g. DCC, EDC) or a uranium coupling agent (e.g. HBTU or, particularly, HATU)), a suitable base (e.g. triethylamine or N,N-diisopropylethylamine) and a suitable solvent (e.g. DCM or DMF) at a suitable temperature (e.g. between 0° C. and 100° C., particularly room temperature).

Compounds of formulae IV, V, VI, VII, X, XI and XII are either commercially available, are known in the literature, and/or may be obtained either by analogy with the processes described herein and/or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

Compounds of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

### BRIEF DESCRIPTION OF THE FIGURES

FIG. 1 shows the results of the passive avoidance task performed with the compound of Example 1 described in Biological Example 3. The graph demonstrates that administering the compound of Example 1 to mice treated with scopolamine improves cognitive function, as illustrated by the increased retention latency within the bright area.

FIG. 2 shows the results of the passive avoidance task performed with the compound of Example 3 described in Biological Example 3. The graph demonstrates that administering the compound of Example 3 to mice treated with scopolamine improves cognitive function, as illustrated by the increased retention latency within the bright area.

FIG. 3 shows the results of the Forced Swim Test described in Biological Example 4. The graph demonstrates that administering the compound of Example 1 to mice decreased time spent immobile compared to mice given the vehicle control. Decreased time spent immobile is a model-specific endpoint commonly used as an indicator of anti-depressant effects in pharmaceutical compounds

### EXAMPLES

The present invention will be further described by reference to the following examples, which are not intended to limit the scope of the invention.

### Experimental Procedures

Starting materials and intermediates used in the synthesis of compounds described herein are commercially available or can be prepared by the methods described herein or by methods known in the art.

Experiments were generally carried out under inert atmosphere (nitrogen or argon), particularly in cases where oxygen- or moisture-sensitive reagents or intermediates were used.

Mass spectrometry data are reported from liquid chromatography-mass spectrometry (LC-MS) using electrospray ionization. Chemical shifts for NMR data are expressed in parts per million (ppm, δ) referenced to residual peaks from the deuterated solvent used.

For syntheses referencing general procedures, reaction conditions (such as length of reaction or temperature) may vary. In general, reactions were followed by thin layer chromatography or LC-MS, and subjected to work-up when appropriate. Purifications may vary between experiments: in general, solvents and the solvent ratios used for eluents/gradients were chosen to provide an appropriate Rf and/or retention time.

### General Methods

All solvents were of analytical grade and commercially available anhydrous solvents were routinely used for reactions. Starting materials used were available from commercial sources or prepared according to literature procedures, Room temperature refers to 20-25° C. Solvent mixture compositions are given as volume percentages or volume ratios. MW heating was performed in a standard MW reactor producing continuous irradiation at 2450 MHz. It is understood that MWs can be used for the heating of reaction mixtures. Typically, an Anton paar microwave synthesizer 300 was used as a microwave synthesizer.

Thin layer chromatography (TLC) was performed on Merck TLC-plates (Silica gel 60 F₂₅₄) and spots were UV visualized. TLC was generally used to monitor reaction progression and solvents used were for example: ethyl acetate or acetonitrile or DCM with 1-10% of MeOH, ethyl acetate with 0-95% hexane. Straight phase flash column chromatography (“flash chromatography”/“column chromatography”) was manually performed on Merck Silica gel 60 (0.040-0.063 mm) or basic aluminum oxide or neutral aluminum oxide, or automatically using ISCO Combiflash® Companion™ system using RediSep™ normal-phase flash columns (“Combiflash”) using the solvent system indicated.

NMR spectra was recorded on a 400 MHz NMR spectrometer (Bruker 400 MHz Avance-III) fitted with a probe of suitable configuration. Spectra were recorded at ambient temperature unless otherwise stated. Chemical fields are given in ppm down- and upfield from TMS (0.00 ppm). The following reference signals were used in ¹H-NMR: TMS δ 0.00, or residual solvent signal of DMSO-d6 δ 2.49, CDCl₃ δ 7.25 (unless otherwise indicated). Resonance multiplicities are denoted s, d, t, q, m, dd, tt, dt br and app for singlet, doublet, triplet, quartet, doublet of doublet, triplet of triplet, doublet of triplet, multiplet, broad and apparent, respectively. In some cases only diagnostic signals are reported.

High pressure liquid chromatography (HPLC) was performed on a reversed phase (RP) column. A gradient was applied using for example mobile phase A (5 mM Ammonium acetate+0.1% Formic acid in water) and B (0.1% Formic acid in Acetonitrile) or A (0.1% NH3 in water) and B (0.1% NH3 in acetonitrile) or A (10 mM Ammonium actetate in water) and B (Acetonitrile).

Reversed phase columns used were for example: BEH C18 (50*2.1 mm), 1.7 μm; X-Bridge C18 (50*4.6 mm), 3.5 μm; X-Bridge/YMCC18 (150*4.6 mm), 5 μm; BEH C18 (50*2.1 mm), 1.7 μm; X-Bridge C8 (250*19) mm, 5 μm. The flowrate used was for example 0.55 ml/min or 1.00 ml/min

Mass spectrometry (MS) analysis were performed in positive and/or negative ion mode using electrospray ionization (ESI+/−).

Preparative HPLC chromatography was run on a Waters e2695 Separation Module with a PDA Detector or on a Shimadzu LC-20AP with an UV detector. Column; X-BRIDGE C18, 150*4.6 mm, 5 μm or X-Bridge C18 (250*19 mm) 5 μm or GEMINI C18 (250*21.2 mm) 5 μm or sunfire c18(150*19) mm, 5 micron or x-bridge c18(150*19) mm, 5 micron or ymc actus triart c18(150*20) mm, 5 micron or kromasil eternity c18 (250*21.2) mm, 5 micron. The flowrate used was for example 10-15 ml/min. The UV spectra were typically recorded at 202 nm & between 214 and 260 nm Lambda max.

A gradient was applied using for example mobile phase A (0.1% NH₃ in water) and B (0.1% NH3 in acetonitrile); A (0.1% TFA in water) and B (Acetonitrile); A (5 mM ammonium bicarbonate+0.05% ammonia in water) and B (Acetonitrile); A (5 mM ammonium bicarbonate) and B (acetonitrile) for LC-separation at a flow rate 1 ml/min.

High pressure liquid chromatography (HPLC) was performed on a straight phase column. A linear gradient or isocratic flow was applied using for example phase A (Hexane) and B (XX)

Compounds have been named using CDD vault from Collaborative Drug Discovery Inc. Burlingame CA, USA or ChemDoodle 8.1.0/9.02 from iChemLabs LLC, USA or ACD/ChemSketch 2012 (14.01) from Advanced Chemistry Development (ACD/labs) Ontario, Canada. In case of inconsistency between a name of a compound and the structural formula of the same compound, it is the structural formula that is decisive for the molecular structure of the compound.

In the event that there is a discrepancy between nomenclature and any compounds depicted graphically, then it is the latter that presides (unless contradicted by any experimental details that may be given or unless it is clear from the context).

### Intermediate 1

### 3-(3-methyl-4-phenoxyphenyl)-1-(3-methylphenyl)urea

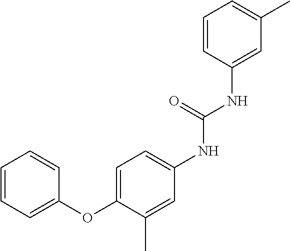 [SMILES: Cc1cccc(NC(=O)Nc2ccc(Oc3ccccc3)c(C)c2)c1]

To a RBF previously equipped with a magnetic stirrer and nitrogen balloon was taken, 3-methyl-4-phenoxyaniline (3.0 g, 15.1 mmol), NaHCO₃ (3.7 g, 45 mmol) and dichloromethane (30 ml). The mixture was cooled to 0° C. and triphosgene (1.33 g, 4.5 mmol) was added and the reaction mixture was stirred for 1 h. 3-methyl aniline (1.6 g, 15.1 mmol) and NaHCO₃ (3.7 g, 45 mmol) were added and the reaction mixture was allowed to reach room temperature and stirred for 3 h. The reaction mixture was quenched with water (50 ml) and the product was extracted with DCM (3×50 ml). The combined organic layer was washed with brine (50 ml), dried over sodium sulphate and the solvent removed under reduced pressure to obtain crude product. The crude product was triturated with DCM and Hexane to obtained 4.8 g (95% yield) of the title compound. MS (ES+) m/z 333 [M+H]⁺

### Intermediate 2

### 1-[3-(methoxymethyl)phenyl]-3-(3-methyl-4-phenoxyphenyl)urea

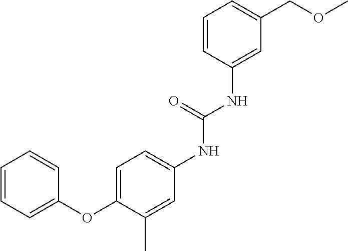 [SMILES: COCc1cccc(NC(=O)Nc2ccc(Oc3ccccc3)c(C)c2)c1]

In a RBF previously equipped with a magnetic stirrer and nitrogen balloon was taken, 3-methyl-4-phenoxyaniline (commercially available, 1.0 g, 5 mmol) and triethylamine (6.96 ml, 50 mmol) in DCM (10 ml) at room temperature and the mixture was stirred for 10 min. Carbonyldiimidazole (CDI, 4.06 g, 25 mmol) was added and the mixture was stirred for 1 hr. 3-methoxymethyl-phenylisocyanate (0.69 g, 5 mmol) was added to the reaction mixture and the mixture was stirred at room temperature for 16 h. The reaction mixture was quenched with ice-water (25 ml) and the product was extracted with DCM (3×25 ml). The combined organic layer was washed with brine (50 ml), dried over sodium sulphate and the solvent removed under reduced pressure to obtain crude product. The crude product was purified by column chromatography using silica gel (100-200 mesh) and 20% ethyl acetate in hexanes as an eluent to obtain 0.55 g (30% yield) of the title compound. MS (ES+) m/z 363 [M+H]⁺

### Intermediate 3

### 2-(methoxymethyl)-4-nitro-1-phenoxybenzene

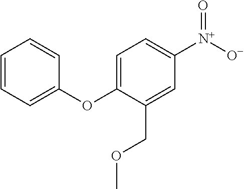 [SMILES: COCc1cc([N+](=O)[O-])ccc1Oc1ccccc1]

In a RBF previously equipped with a magnetic stirrer and nitrogen balloon was taken, Phenol (1.14 g, 12.2 mmol) and K₂CO₃ (3.36 g, 24.3 mmol) in N-Methyl-2-pyrrolidone (15 ml) and stirred for 1 h. 1-Fluoro-2-(methoxymethyl)-4-nitrobenzene (1.5 g, 8.1 mmol) was added in to the mixture and the reaction mixture was heated to 100° C. and stirred for 16 h. The reaction mixture was quenched with water (50 ml) and product was extracted with EtOAc (3×50 ml). The combined organic layer was washed with brine (50 ml), dried over sodium sulphate and the solvent removed under reduced pressure. The crude product was purified by column chromatography using silica gel (100-200 mesh) and 5% ethyl acetate in hexanes as an eluent to obtain 1.6 g (76% yield) of the title compound. ¹H NMR (400 MHz, DMSO-d₆): δ 8.29 (d, J=2.40 Hz, 1H), 8.15 (dd, J=2.80, 9.20 Hz, 1H), 7.49 (app t, 2H), 7.30 (t, J=7.20 Hz, 1H), 7.16 (app d, 2H), 6.85 (d, J=9.20 Hz, 1H), 4.62 (s, 2H), 3.43 (s, 3H). MS (ES+) m/z 260 [M+H]⁺

### Intermediate 4

### 3-(methoxymethyl)-4-phenoxyaniline

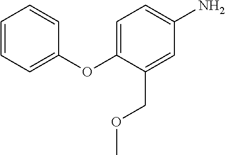 [SMILES: COCc1cc(N)ccc1Oc1ccccc1]

To a RBF previously equipped with a magnetic stirrer and nitrogen balloon was added 2-(methoxymethyl)-4-nitro-1-phenoxybenzene (Intermediate 3, 1.6 g, 8.0 mmol) in Methanol (16 ml). 10% Pd/C (50% wet, 0.32 g) was added under N₂ atmosphere. The suspension was stirred under H₂ (gas) bubbling. The completion of reaction was confirmed by the TLC using Hexane:EtOAc (7:3) as mobile phase. The TLC was visualized using UV light. After completion of the reaction, reaction mixture was filtered through celite bed washed with methanol and solvent was removed under reduced pressure to obtain 1.4 g (97% yield) of the title compound. ¹H NMR (400 MHz, DMSO-d₆): δ 7.28 (app t, 2H), 6.97 (t, J=6.80 Hz, 1H), 6.79 (app d, 2H), 6.68 (s, 2H), 6.51 (d, J=8.40 Hz, 1H), 5.05 (s, 2H), 4.20 (s, 2H), 3.20 (s, 3H). MS (ES+) m/z 230 [M+H]⁺

### Intermediate 5

### 3-[3-(methoxymethyl)-4-phenoxyphenyl]-1-(3-methylphenyl)urea

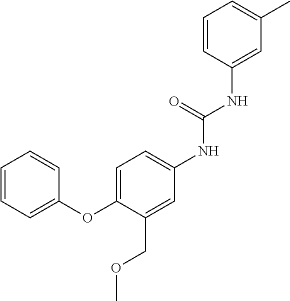 [SMILES: COCc1cc(NC(=O)Nc2cccc(C)c2)ccc1Oc1ccccc1]

In a RBF previously equipped with a magnetic stirrer and nitrogen balloon was taken 3-(methoxymethyl)-4-phenoxyaniline, (Intermediate 4, 0.35 g, 1.5 mmol) and NaHCO₃ (0.38 g, 4.5 mmol) in DMF (3.5 ml) and the mixture was cooled to 0° C. 3-methyl-phenylisocyanate (0.203 g, 1.5 mmol) was added and the resulting reaction mixture was allowed to reach 25° C. and stirred for 3 h. The reaction mixture was quenched with ice-water (50 ml) and the solid precipitate obtained was collected by filtration, washed with water (50 ml) and dried under vacuum to yield 0.4 g (72% yield) of the title compound, that was used in the next step without further purification. ¹H NMR (400 MHz, DMSO-d6): δ 8.74 (s, 1H), 8.55 (s, 1H), 7.62 (s, 1H), 7.32-7.34 (m, 4H), 7.22 (d, J=7.60 Hz, 1H), 7.15 (t, J=7.60 Hz, 1H), 7.06 (t, J=7.20 Hz, 1H), 6.88 (app d, 3H), 6.78 (d, J=7.2 Hz, 1H), 4.35 (s, 2H), 3.27 (s, 3H), 2.27 (s, 3H). MS (ES+) m/z 363 [M+H]⁺

### Intermediate 6

### Phenyl N-(2-methyl-1-benzofuran-7-yl)carbamate

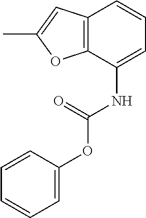 [SMILES: Cc1cc2cccc(NC(=O)Oc3ccccc3)c2o1]

In a RBF previously equipped with a magnetic stirrer and nitrogen balloon was taken 2-Methyl-1-benzofuran-7-ylamine (commercially available, 0.8 g, 5.3 mmol) and K₂CO₃(2.19 g, 16 mmol) in THF (16 ml) at 25° C. and the mixture was stirred for 30 min. Phenyl chloroformate (1.25 g, 8 mmol) was added drop wise in to the reaction mixture and it was stirred at 25° C. for 16 h. The reaction mixture was quenched with ice-water (30 ml) and the product was extracted with Ethyl Acetate (3×20 ml). The combined organic layer was washed with brine (30 ml). The organic layer was dried over sodium sulphate and the solvent removed under reduced pressure to obtain 1.44 g (99%) of the title compound. MS (ES+) m/z 268 [M+H]⁺

### Intermediate 7

### 3-(2-methyl-1-benzofuran-7-yl)-1-(3-methyl-4-phenoxyphenyl)urea

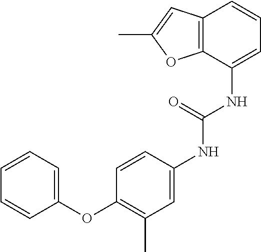 [SMILES: Cc1cc2cccc(NC(=O)Nc3ccc(Oc4ccccc4)c(C)c3)c2o1]

In a seal tube previously equipped with a magnetic stirrer was taken Phenyl N-(2-methyl-1-benzofuran-7-yl)carbamate (Intermediate 6, 1.00 g 3.7 mmol) and 3-methyl-4-phenoxyaniline (commercially available, 0.73 g, 3.7 mmol) in DMF (10 ml) at 25° C. Triethylamine (0.74 g, 7.4 mmol) was added and the reaction mixture was heated to 100° C. and stirred for 16 h. The reaction mixture was quenched with ice-water (40 ml) and the product was extracted with ethyl acetate (3×25 ml). The combined organic layer was washed with brine (30 ml). The organic layer was dried over sodium sulphate and the solvent removed under reduced pressure. The crude product was purified by column chromatography using 20% Ethyl acetate in Hexane as a mobile phase and 60-120 silica on column chromatography to yield 0.26 g (18% yield) of the title compound. MS (ES+) m/z 373 [M+H]⁺

### Intermediate 8

### methyl 5-{[(3-methylphenyl)carbamoyl]amino}-2-phenoxybenzoate

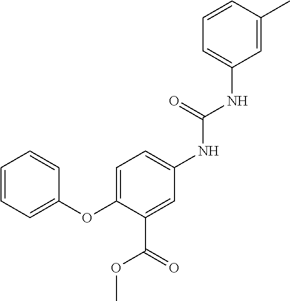 [SMILES: COC(=O)c1cc(NC(=O)Nc2cccc(C)c2)ccc1Oc1ccccc1]

To a RBF previously equipped with a magnetic stirrer and nitrogen balloon was added, 3-methylaniline (1.35 g, 12.6 mmol), NaHCO₃ (3.17 g, 37.8 mmol) and DCM (17 ml). The mixture was cooled to 0° C., and triphosgene (1.23 g, 4.1 mmol) was added and reaction mixture was stirred for 2 h at 0° C. Methyl 5-amino-2-phenoxybenzoate (commercially available, 3.08 g, 12.6 mmol) and NaHCO₃ (3.17 g, 37.8 mmol) were added to the reaction mixture. The reaction mixture was allowed to reach to 25° C. and stirred for 2 h. The reaction mixture was quenched with water (50 ml) and the product was extracted with DCM (3×50 ml). The combined organic layer was washed with brine (50 ml), dried over sodium sulphate and the solvent removed under reduced pressure and the crude product was purified by column chromatography using silica gel (60-120 mesh) and 30% ethyl acetate in hexanes as an eluent to obtain 4.5 g (94% yield) of the title compound. MS (ES+) m/z 377 [M+H]⁺

### Intermediate 9

### 3-[3-(hydroxymethyl)-4-phenoxyphenyl]-1-(3-methylphenyl)urea

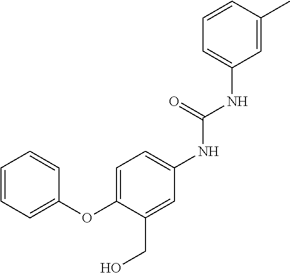 [SMILES: Cc1cccc(NC(=O)Nc2ccc(Oc3ccccc3)c(CO)c2)c1]

To a RBF previously equipped with a magnetic stirrer and nitrogen balloon was added, methyl 5-{[(3-methylphenyl)carbamoyl]amino}-2-phenoxybenzoate (Intermediate 8, 4.50 g, 11.9 mmol) in THF (45 ml) and the mixture was cooled to 0° C. LiBH₄ (1.56 g, 71.7 mmol) was added portion wise at 0° C. The reaction mixture was allowed to reach room temperature and stirred for 4 h. The reaction mixture was quenched with water (50 ml) and the product was extracted with ethylacetate (3×50 ml), dried over sodium sulphate and the solvent removed under reduced pressure. The crude product was triturated by DCM (2×15 ml) and the solid product was collected by filtration to yield 3.9 g (93%) of the title compound. ¹H NMR (400 MHz, DMSO-d₆): δ 8.76 (s, 1H), 8.57 (s, 1H), 7.64 (s, 1H), 7.35-7.40 (m, 4H), 7.33-7.15 (m, 2H), 7.05-7.06 (m, 1H), 6.81-6.79 (m, 4H), 5.21 (d, J=4.80 Hz, 1H), 4.45 (d, J=4.80 Hz, 2H), 2.30 (s, 3H). MS (ES+) m/z 349 [M+H]⁺

### Intermediate 10

### 3-[3-(chloromethyl)-4-phenoxyphenyl]-1-(3-methylphenylurea

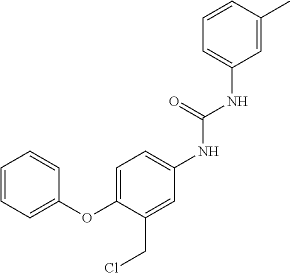 [SMILES: Cc1cccc(NC(=O)Nc2ccc(Oc3ccccc3)c(CCl)c2)c1]

To a RBF previously equipped with a magnetic stirrer and nitrogen balloon was added 3-[3-(hydroxymethyl)-4-phenoxyphenyl]-1-(3-methylphenyl)urea (Intermediate 9, 3.80 g, 10.9 mmol) in dichloromethane (38 ml). A catalytic amount of DMF (0.5 ml) was added and the mixture was cooled to 0° C. and stirred for 10 min at 0° C. Thionyl chloride (2.59 g, 21.8 mmol) was added drop wise and the resulting reaction mixture was allowed to reach room temperature and stirred for 2 h. The reaction mixture was quenched with water (30 ml) and the aqueous layer was extracted with dichloromethane (3×30 ml). The combined organic layer was dried over sodium sulphate and the solvent removed under reduced pressure. The crude product was purified by column chromatography using silica gel (100-200 mesh) and neat hexanes as an eluent to obtain 3.0 g (74% yield) of the title compound. MS (ES+) m/z 367 [M+H]⁺

### Intermediate 11

### 1-[3-(chloromethyl)-4-phenoxyphenyl]-3-(3-methylphenyl)-1,3,5-triazinane-2,4,6-trione

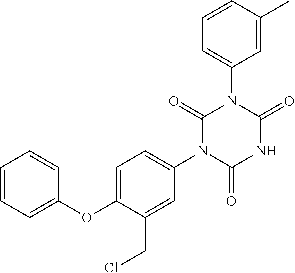 [SMILES: Cc1cccc(-n2c(=O)[nH]c(=O)n(-c3ccc(Oc4ccccc4)c(CCl)c3)c2=O)c1]

To a microwave vial previously equipped with a magnetic stirrer and nitrogen balloon was added 3-[3-(chloromethyl)-4-phenoxyphenyl]-1-(3-methylphenyl)urea (Intermediate 10, 1.00 g, 2.3 mmol) in bromobenzene (10 ml) and the mixture was cooled to 0° C. Ethoxy carbonyl isocyanate (0.53 g, 4.5 mmol) was added and resulting reaction mixture was allowed to reach 25° C. and heated at 150° C. for 3 h in an Anton par microwave synthesizer-300. The reaction mixture was quenched with water (10 ml) and aqueous layer was extracted with ethyl acetate (2×30 ml). The combined organic layer was dried over sodium sulphate and the solvent removed under reduced pressure. The crude product was purified by Combi-flash chromatography and 20% ethyl acetate in hexanes as an eluent to obtain 0.53 g of the crude title compound that was used in the next step without further purification. MS (ES-) m/z 434 [M−H]⁻

### Intermediate 12

### 1-(5-nitro-2-phenoxyphenyl)ethan-1-one

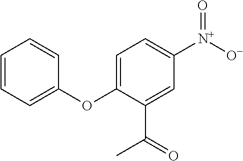 [SMILES: CC(=O)c1cc([N+](=O)[O-])ccc1Oc1ccccc1]

In a sealed tube, previously equipped with a magnetic stirrer and nitrogen balloon, was taken phenol (7.39 g, 78.6 mmol) in o-Xylene (150 ml) at room temperature and K₂CO₃(12.07 g, 87.3 mmol) was added. The mixture was stirred for 1 h at room temperature and 1-(2-fluoro-5-nitrophenyl)ethan-1-one (commercially available, 8.0 g, 43.6 mmol) in o-Xylene (42 ml) was added and the reaction mixture was stirred at 140° C. for 16 h. The reaction mixture was quenched with ice-water (300 ml) and the product was extracted with Ethyl Acetate (3×200 ml). The combined organic layer was washed with brine (300 ml), dried over sodium sulphate and the solvent removed under reduced pressure. The obtained crude product was triturated by dichloromethane:Hexane (1:9; 100 ml) to obtain 7.92 g (70% yield) of the title compound. 1H NMR (400 MHz, DMSO-d6): δ 8.49 (s, 1H), 6.33-7.31 (m, 1H), 7.52 (s, 2H), 7.33-7.28 (m, 3H), 6.98 (d, J=8.0 Hz, 1H), 2.67 (s, 3H).

### Intermediate 13

### 2-ethyl-4-nitro-1-phenoxybenzene

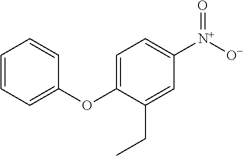 [SMILES: CCc1cc([N+](=O)[O-])ccc1Oc1ccccc1]

In a RBF previously equipped with a magnetic stirrer was taken 1-(5-nitro-2-phenoxyphenyl)ethan-1-one (Intermediate 12, 7.92 g, 30.8 mmol) in TFA (159 ml) and the mixture was purged with nitrogen gas for 10 minutes. Et₃SiH (14.33 g, 123.2 mmol) and LiClO₄ (0.034 g, 0.31 mmol) were added under N₂ atmosphere. The reaction mixture was stirred at RT for 6 h. The solvent was removed under reduced pressure to obtain 12.0 g of the crude title compound that was used without further purification in the next step. MS (ES+) m/z 244 [M+H]⁺

### Intermediate 14

### 3-ethyl-4-phenoxyaniline

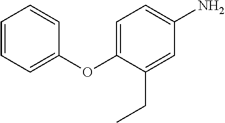 [SMILES: CCc1cc(N)ccc1Oc1ccccc1]

In a RBF previously equipped with a magnetic stirrer and nitrogen balloon was taken 2-ethyl-4-nitro-1-phenoxybenzene (Intermediate 13, 12 g, 49.3 mmol) in methanol (150 ml). 10% Pd/C (3.0 g, 50% wet) was added under N₂ atmosphere. The suspension was stirred for 6 h under H₂ (gas) bubbling. The reaction mixture was filtered through a celite pad and the solvent removed under reduced pressure. The obtained crude was purified by column chromatography (30% Ethyl acetate in hexanes) to obtain 4.5 g (68%) of the title compound. 1H NMR (400 MHz, DMSO-d6): δ 7.26 (app t, J=8.00 Hz, 2H), 6.95 (t, J=7.20 Hz, 1H), 6.78 (app d, J=8.00 Hz, 2H), 6.66 (d, J=8.40 Hz, 1H), 6.52 (d, J=2.00 Hz, 1H), 6.43-6.45 (m, 1H), 4.95 (s, 2H), 2.34 (q, J=7.20 Hz, 2H), 1.03 (t, J=7.20 Hz, 3H). MS (ES+) m/z 214 [M+H]⁺

### Intermediate 15

### 3-(3-ethyl-4-phenoxyphenyl)-1-phenylurea

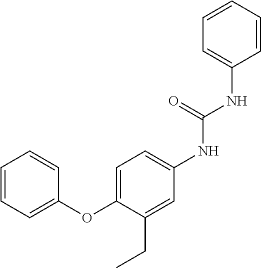 [SMILES: CCc1cc(NC(=O)Nc2ccccc2)ccc1Oc1ccccc1]

In a RBF previously equipped with a magnetic stirrer and nitrogen balloon was taken 3-ethyl-4-phenoxyaniline (Intermediate 14, 1.20 g, 5.6 mmol) in DMF (12.0 ml) and the mixture was cooled to 0° C. NaHCO₃ (1.418 g, 16.4 mmol) was added and the resulting mixture was stirred for 20 min at 0° C. Phenyl isocyanate (0.804 g, 6.7 mmol) was added and the reaction mixture was stirred at RT for 4 h. The reaction mixture was quenched with water (30 ml) and the product was extracted with EtOAc (3×30 ml). The combined organic layer was washed with brine (20 ml), dried over sodium sulphate and the solvent removed under reduced pressure to obtain 1.21 g (64%) of the title compounds that was used in the next step without further purification. MS (ES+) m/z 333 [M+H]⁺

### Intermediate 16

### 3-(3-ethyl-4-phenoxyphenyl)-1-(3-methylphenyl)urea

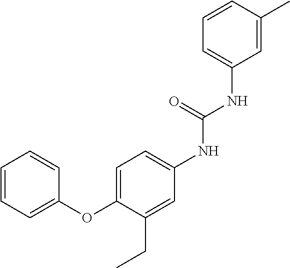 [SMILES: CCc1cc(NC(=O)Nc2cccc(C)c2)ccc1Oc1ccccc1]

In a RBF previously equipped with a magnetic stirrer and nitrogen balloon was taken 3-ethyl-4-phenoxyaniline (Intermediate 14, 1.20 g, 5.6 mmol) in DMF (12.0 ml) and the mixture was cooled to 0° C. NaHCO₃ (1.418 g, 16.8 mmol) was added and the resulting mixture was stirred for 20 min at 0° C. 3-Methyl-phenyl isocyanate (0.898 g, 6.7 mmol) was added and the reaction mixture stirred at RT for 4 h. The reaction mixture was quenched with water (30 ml) and the product was extracted with EtOAc (3×30 ml). The combined organic layer was washed with brine (20 ml), dried over sodium sulphate and the solvent removed under reduced pressure to obtain 1.22 g (62%) of the title compound that was used in the next step without further purification. MS (ES+) m/z 347 [M+H]⁺

### Intermediate 17

### N-cyano-N′-(3-methyl-4-phenoxyphenyl)(methylsulfanyl)methanimidamide

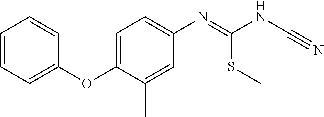 [SMILES: CS/C(=N\c1ccc(Oc2ccccc2)c(C)c1)NC#N]

In a RBF previously equipped with a magnetic stirrer and nitrogen balloon was taken 3-methyl-4-phenoxyaniline (commercially available, 2.0 g, 10 mmol) in Ethanol (30 ml). To this solution dimethyl N-cyanodithioiminocarbonate (1.47 g, 10 mM) was added at room temperature. The reaction mixture was heated to 80° C. and stirred for 72 h. The reaction mixture was cooled to room temperature and the solvent was removed under reduced pressure. The crude product was purified by Combi-flash chromatography using 50% ethyl acetate in hexanes as an eluent to obtain 2.2 g (73% yield) of the title compound. ¹H NMR (400 MHz, DMSO-d₆): δ 10.14 (s, 1H), 7.40-7.36 (m, 3H), 7.29 (d, J=8.4 Hz, 1H), 7.11 (t, J=7.2 Hz, 1H), 6.92 (app d, J=8.4 Hz, 3H), 2.70 (s, 3H), 2.18 (s, 3H). MS (ES+) m/z 298 [M+H]⁺

### Intermediate 18

### 1-(4-methoxyphenyl)-3-[1-(4-methoxyphenyl)-5-(3-methyl-4-phenoxyphenyl)-4-(methylsulfanyl)-6-oxo-1,2,5,6-tetrahydro-1,3,5-triazin-2-ylidene]urea

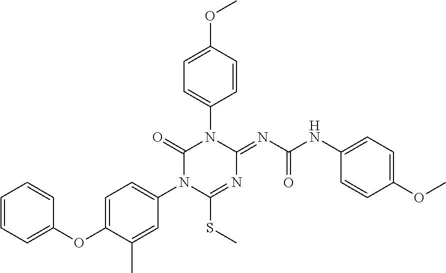 [SMILES: COc1ccc(NC(=O)/N=c2\nc(SC)n(-c3ccc(Oc4ccccc4)c(C)c3)c(=O)n2-c2ccc(OC)cc2)cc1]

In a 25 ml RBF previously equipped with a magnetic stirrer was taken N-cyano-N′-(3-methyl-4-phenoxyphenyl)(methylsulfanyl)methanimidamide (Intermediate 17, 0.40 g, 1.3 mmol) and 4-methoxy-phenyl isocyanate (0.34 g, 2.3 mmol) in DCM (8.0 ml). To the mixture, triethylamine (1.90 ml, 13.4 mmol) was added followed by portion wise (3-4 portions) addition of 1,1′-Carbonyldiimidazole (1.10 g, 6.7 mmol). The reaction mixture was stirred at room temperature for 6 h. The reaction mixture was quenched with water (30 ml) and both the layers were separated. Aqueous layer was extracted with DCM (2×30 ml). The combined organic layer was washed with brine (30 ml), dried over sodium sulphate and the solvent removed under reduced pressure to obtain crude product that was purified by Combi-flash chromatography using 50% ethyl acetate in hexanes as an eluent to obtain 0.45 g of the title compound that was used in the next step without further purification. MS (ES+) m/z 596 [M+H]⁺

### Intermediate 19

### 3-(3-hydroxy-4-phenoxyphenyl)-1-(3-methylphenyl)urea

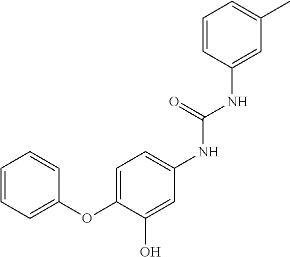 [SMILES: Cc1cccc(NC(=O)Nc2ccc(Oc3ccccc3)c(O)c2)c1]

To a RBF previously equipped with a magnetic stirrer and nitrogen balloon was added 5-Amino-2-phenoxyphenol (commercially available, 2.0 g, 9.9 mmol) in DMF (20 ml) and the mixture was cooled to 0° C. NaHCO₃ (2.49 g, 29.7 mmol) was added and the resulting mixture was stirred for 20. 3-Methyl-phenylisocyanate (1.32 g, 9.9 mmol) was added and reaction mixture was stirred at room temperature for 4 h. The reaction mixture was quenched with water (500 ml) and product was extracted with EtOAc (3×100 ml). The combined organic layer was washed with brine (100 ml), dried over sodium sulphate and the solvent removed under reduced pressure. The crude product was triturated with dichloromethane and hexane to obtain 3.5 g of the title compound. MS (ES+) m/z 335 [M+H]⁺

### Intermediate 20

### ethyl 2-(5-{[(3-methylphenyl)carbamoyl]amino}-2-phenoxyphenoxy)acetate

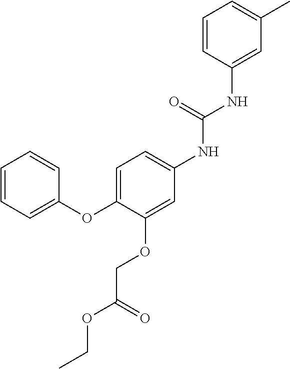 [SMILES: CCOC(=O)COc1cc(NC(=O)Nc2cccc(C)c2)ccc1Oc1ccccc1]

In a RBF previously equipped with a magnetic stirrer and nitrogen balloon was taken 3-(3-hydroxy-4-phenoxyphenyl)-1-(3-methylphenyl)urea (Intermediate 19, 3.00 g, 8.97 mmol) and K₂CO₃ (1.85 g, 13.46 mmol) in DMF (30 ml) and the mixture was cooled to O ° C. To the reaction mixture, BrCH₂OOEt (2.24 g, 13.46 mmol) was added and the resulting reaction mixture was stirred for 16 h at room temperature. The reaction mixture was diluted with ice cold water (100 ml) and extracted with EtOAc (3×50 ml). The combined organic layer was washed with brine (100 ml), dried over sodium sulphate and the solvent removed under reduced pressure. The crude product was purified by column chromatography using silica gel (60-120 mesh) and 20% ethyl acetate in hexane as an eluent to obtain 3.5 g of the title compound. MS (ES+) m/z 421 [M+H]⁺

### Intermediate 21

### ethyl 2-{5-[3-(3-methylphenyl)-2,4,6-trioxo-1,3,5-triazinan-1-yl]-2-phenoxyphenoxy}acetate

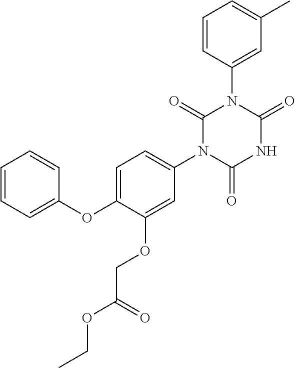 [SMILES: CCOC(=O)COc1cc(-n2c(=O)[nH]c(=O)n(-c3cccc(C)c3)c2=O)ccc1Oc1ccccc1]

In a sealed tube previously equipped with a magnetic stirrer and nitrogen balloon was taken ethyl 2-(5-{[(3-methylphenyl)carbamoyl]amino}-2-phenoxyphenoxy)acetate (Intermediate 20, 3.50 g, 8.32 mmol) in Chlorobenzene (35 ml) and the mixture was cooled to 0° C. Ethoxy carbonyl isocyanate (3.83 g, 33.3 mmol) was added drop-wise and the resulting reaction mixture was allowed to reach room temperature and heated at 150° C. for 16 h. The solvent was removed under reduced pressure and the crude product was purified by column chromatography using silica gel (100-200 mesh) and 70% ethyl acetate in hexanes as an eluent to obtain 1.071 g of the crude title compound that was used in the next step without further purification. MS (ES-) m/z 488 [M−H]⁻.

### Intermediate 22

### 2-{5-[3-(3-methylphenyl)-2,4,6-trioxo-1,3,5-triazinan-1-yl]-2-phenoxyphenoxy}acetic acid

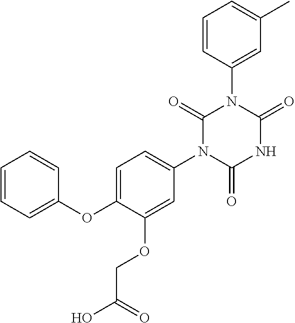 [SMILES: Cc1cccc(-n2c(=O)[nH]c(=O)n(-c3ccc(Oc4ccccc4)c(OCC(=O)O)c3)c2=O)c1]

In a RBF previously equipped with a magnetic stirrer was taken ethyl 2-{5-[3-(3-methylphenyl)-2,4,6-trioxo-1,3,5-triazinan-1-yl]-2-phenoxyphenoxy}acetate (Intermediate 21, 1.0 g) in THF:H₂O (8 ml: 2 ml). LiOH (1.85 g, 13.4 mmol) was added and the resulting reaction mixture was stirred for 1 h at RT. The reaction mixture was concentrated under reduced pressure and the crude mass was diluted with water (5 ml) and extracted with EtOAc (2×10 ml). The aqueous layer was acidified using 1M HCl to pH-3-4 and then again extracted with EtOAc (3×20 ml). The combined organic layer was dried over sodium sulphate and the solvent removed under reduced pressure. The crude product was triturated with DCM and Hexane to obtain the 0.8 g of the title compound. MS (ES-) m/z 460 [M−H]⁻.

### Intermediate 23

### 2-(methylsulfanyl)-4-nitro-1-phenoxybenzene

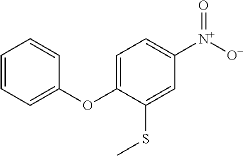 [SMILES: CSc1cc([N+](=O)[O-])ccc1Oc1ccccc1]

In a seal tube previously equipped with a magnetic stirrer and nitrogen balloon was taken Phenol (0.84 g, 8.97 mmol) and Cs₂CO₃ (2.92 g, 8.97 mmol) in DMSO (84 ml) and the mixture was cooled to 0° C. To the reaction mixture, 1-fluoro-2-(methylsulfanyl)-4-nitrobenzene (commercially available, 1.4 g, 7.48 mmol) was added and the resulting reaction mixture was allowed to reach room temperature and then stirred at 110° C. for 5 h. The reaction mixture was quenched with water (50 ml) and product was extracted with EtOAc (3×40 ml). The combined organic layer was dried over sodium sulphate and the solvent removed under reduced pressure to obtain crude product that was purified by column chromatography using silica gel (60-120 mesh) and 10% ethyl acetate in hexane as an eluent to obtain 1.0 g (51% yield) of the title compound. ¹H NMR (400 MHz, DMSO-d₆): δ 8.06-8.01 (m, 2H), 7.50 (app t, 2H), 7.30 (t, J=7.20 Hz, 1H), 7.15 (app d, 2H), 6.89 (d, J=9.20 Hz, 1H), 2.60 (s, 3H).

### Intermediate 24

### 3-(methylsulfanyl)-4-phenoxyaniline

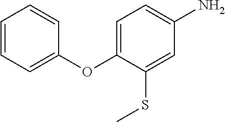 [SMILES: CSc1cc(N)ccc1Oc1ccccc1]

In a RBF previously equipped with a magnetic stirrer and nitrogen balloon was taken 3-(methylsulfanyl)-4-phenoxyaniline (Intermediate 23, 0.50 g, 1.91 mmol) in Methanol (5.0 ml). 10% Pd/C (50% wet, 0.101 g, 0.956 mmol) was added under N₂ atmosphere. The mixture was stirred under H₂ (gas) bubbling. The completion of reaction was confirmed by the TLC using Hexane:EtOAc (8:2) as mobile phase and monitored using TLC (visualized using UV light). After completion, the reaction mixture was filtered through a celite bed, washed with methanol and the solvent was removed under reduced pressure to obtain 0.400 g (90%) of the title compound. MS (ES+) m/z 232 [M+H]⁺

### Intermediate 25

### 3-[3-(methylsulfanyl)-4-phenoxyphenyl]-1-phenylurea

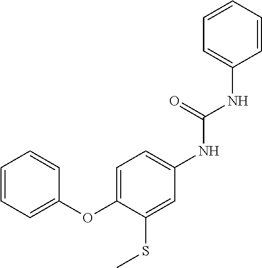 [SMILES: CSc1cc(NC(=O)Nc2ccccc2)ccc1Oc1ccccc1]

In a RBF previously equipped with a magnetic stirrer and nitrogen balloon was taken 3-(methylsulfanyl)-4-phenoxyaniline (Intermediate 24, 0.400 g, 1.729 mmol) in DMF (4.0 ml) and the mixture was cooled to 0° C. To the reaction mixture, NaHCO₃ (0.435 g, 5.187 mmol) was added and the resulting mixture was stirred for 20 min at 0° C. Phenyl isocyanate (0.205 g, 1.729 mmol) was added and the reaction stirred at RT for 3 h. The reaction mixture was quenched with water (30 ml) and the product was extracted with EtOAc (3×30 ml). The combined organic layer was washed with brine (20 ml), dried over sodium sulphate and the solvent removed under reduced pressure to obtain the crude product that was purified by column chromatography using Hexanes:ethyl acetate as an eluent using 60-120 mesh silica gel to yield 0.280 g (46%) of the title compound. MS (ES+) m/z 351 [M+H]⁺

### Intermediate 26

### 1-(3-methylphenyl)-3-[3-(methylsulfanyl)-4-phenoxyphenyl]urea

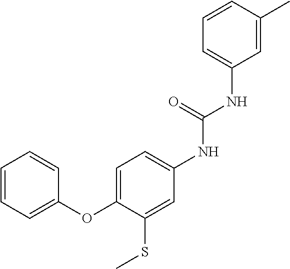 [SMILES: CSc1cc(NC(=O)Nc2cccc(C)c2)ccc1Oc1ccccc1]

In a RBF previously equipped with a magnetic stirrer and nitrogen balloon was taken, 3-methyl-aniline (0.185 g, 1.729 mmol), NaHCO₃ (0.435 g, 5.187 mmol) and DCM (12 ml). The mixture was cooled to 0° C. and Triphosgene (0.168 g, 0.570 mmol) was added and the reaction mixture was stirred for 1 h. After that, 3-(methylsulfanyl)-4-phenoxyaniline (Intermediate 24, 0.400 g, 1.729 mmol) and NaHCO₃ (0.435 g, 5.187 mmol) were added and the reaction mixture was allowed to reach room temperature. The reaction mixture was stirred at room temperature for 3 h. The reaction mixture was quenched with water (50 ml) and the product was extracted with EtOAc (3×50 ml). The combined organic layer was washed with brine (50 ml), dried over sodium sulphate and the solvent removed under reduced pressure to obtain crude product. The crude product was purified by column chromatography using silica gel (60-120 mesh) and 2% methanol in Dichloromethane as an eluent to obtain 0.320 g (50% yield) of the title compound. MS (ES+) m/z 365 [M+H]⁺.

### Example 1

### 1-(3-methyl-4-phenoxyphenyl)-3-(3-methylphenyl)-1,3,5-triazinane-2,4,6-trione

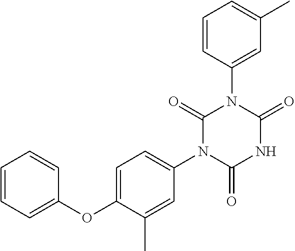 [SMILES: Cc1cccc(-n2c(=O)[nH]c(=O)n(-c3ccc(Oc4ccccc4)c(C)c3)c2=O)c1]

In a microwave tube previously equipped with a magnetic stirrer and nitrogen balloon was taken 3-(3-methyl-4-phenoxyphenyl)-1-(3-methylphenyl)urea (Intermediate 1, 2.8 g, 8.4 mmol) in Bromobenzene (28 ml) and the mixture was cooled to 0° C. Ethoxy carbonyl isocyanate (3.88 g, 33.7 mmol) was added and resulting reaction mixture was allowed to come to 25° C. and then heated at 150° C. for 16 h. The reaction mixture was quenched with water (10 ml) and the aqueous layer was extracted with ethyl acetate (2×30 ml). The combined organic layer was dried over sodium sulphate and the solvent removed under reduced pressure to obtain the crude product that was purified by preparative RP-HPLC (acetonitrile 25-100% in water [5 mM Ammonium bicarbonate+0.1% NH3]) to yield 89 mg, (26% yield) of the title compound. ¹H NMR (400 MHz, DMSO-d₆): δ 12.03 (s, 1H), 7.43-7.33 (m, 4H), 7.25-7.13 (m, 5H), 6.99 (s, 1H), 6.97 (s, 1H), 6.93 (d, J=8.4 Hz, 1H), 2.35 (s, 3H), 2.22 (s, 3H). MS (ES-) m/z 400 [M−H]⁻.

### Example 2

### 1-[3-(methoxymethyl)phenyl]-3-(3-methyl-4-phenoxyphenyl)-1,3,5-triazinane-2,4,6-trione

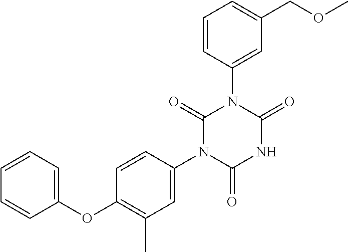 [SMILES: COCc1cccc(-n2c(=O)[nH]c(=O)n(-c3ccc(Oc4ccccc4)c(C)c3)c2=O)c1]

In a microwave tube previously equipped with a magnetic stirrer and nitrogen balloon was taken 1-[3-(methoxymethyl)phenyl]-3-(3-methyl-4-phenoxyphenyl)urea (Intermediate 2, 0.5 g, 1.4 mmol) in Chlorobenzene (5 ml) and the mixture was cooled to 0° C. Ethoxy carbonyl isocyanate (0.644 g, 5.6 mmol) was added and resulting reaction mixture was allowed to reach room temperature and then heated at 150° C. for 3 h in Anton paar microwave synthesizer-300. The solvent was removed under reduced pressure to obtain that crude product, that was purified by preparative RP-HPLC (acetonitrile 20-100% in water [5 mM Ammonium bicarbonate+0.1% NH3]) to yield 0.055 g (9% yield) of the title compound. ¹H NMR (400 MHz, DMSO-d6): δ 12.00 (s, 1H), 7.46-7.27 (m, 7H), 7.2 (d, J=8.0, 1H), 7.13 (t, J=7.2 Hz, 1H), 6.89-7.01 (m, 3H), 4.45 (s, 2H), 3.49 (s, 3H), 2.21 (s, 3H). MS (ES-) m/z 430 [M−H]⁻.

### Example 3

### 1-[3-(methoxymethyl)-4-phenoxyphenyl]-3-(3-methylphenyl)-1,3,5-triazinane-2,4,6-trione

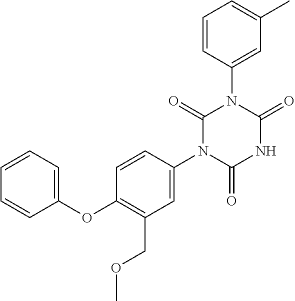 [SMILES: COCc1cc(-n2c(=O)[nH]c(=O)n(-c3cccc(C)c3)c2=O)ccc1Oc1ccccc1]

In a microwave tube previously equipped with a magnetic stirrer and nitrogen balloon was taken 3-[3-(methoxymethyl)-4-phenoxyphenyl]-1-(3-methylphenyl)urea (Intermediate 5, 0.2 g, 0.6 mmol) in Chlorobenzene (2 ml) and the mixture was cooled to 0° C. Ethoxy carbonyl isocyanate (0.191 g, 1.7 mmol) was added and the resulting reaction mixture was allowed to reach room temperature and heated at 150° C. for 3 h in an Anton par microwave synthesizer-300. The solvent was removed under reduced pressure and the crude product was purified by preparative RP-HPLC (acetonitrile 35-100% in water [0.1% formic acid]) to yield 0.022 g (9% yield) of the title compound. ¹H NMR (400 MHz, DMSO-d₆): δ 11.98 (s, 1H), 7.49 (s, 1H), 7.42 (t, J=7.60 Hz, 2H), 7.35 (t, J=7.60 Hz, 1H), 7.27 (dd, J=2.00, 8.60 Hz, 1H), 7.23 (d, J=7.60 Hz, 1H), 7.15-7.18 (m, 3H), 7.02 (d, J=7.60 Hz, 2H), 6.89 (d, J=8.40 Hz, 1H), 4.49 (s, 2H), 3.34 (s, 3H), 2.34 (s, 3H). MS (ES-) m/z 430 [M−H]⁻.

### Example 4

### 1-(2-methyl-1-benzofuran-7-yl)-3-(3-methyl-4-phenoxyphenyl)-1,3,5-triazinane-2,4,6-trione

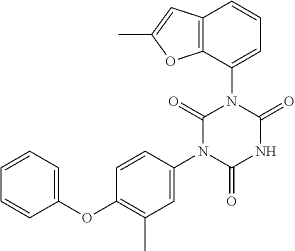 [SMILES: Cc1cc2cccc(-n3c(=O)[nH]c(=O)n(-c4ccc(Oc5ccccc5)c(C)c4)c3=O)c2o1]

In a 10 ml microwave vial previously equipped with a magnetic stirrer and nitrogen balloon was taken 3-(2-methyl-1-benzofuran-7-yl)-1-(3-methyl-4-phenoxyphenyl)urea (Intermediate 7, 0.210 g, 0.5 mmol) in Bromobenzene (2.1 ml) and the mixture was cooled to 0° C. Ethoxy carbonyl isocyanate (0.227 g, 1.9 mmol) was added drop wise and resulting reaction mixture was allowed to reach room temperature and heated at 150° C. for 3 h in Anton paar microwave synthesizer-300. The solvent was removed under reduced pressure to obtain crude product. The crude product was purified by preparative RP—H PLC (acetonitrile 55-100% in water [0.1% formic acid]) to yield 30 mg (12% yield) of the title compound. ¹H NMR (400 MHz, DMSO-d6): δ 12.22 (s, 1H), 7.61-7.58 (m, 1H), 7.41-7.37 (m, 3H), 7.28-7.26 (m, 3H), 7.13 (t, J=7.20 Hz, 1H), 6.99 (m, 3H), 6.68 (s, 1H), 2.45 (s, 3H), 2.22 (s, 3H). MS (ES-) m/z 440 [M−H]⁻.

### Example 5

### 1-(3-methylphenyl)-3-{3-[(2-oxoimidazolidin-1-yl)methyl]-4-phenoxyphenyl}-1,3,5-triazinane-2,4,6-trione

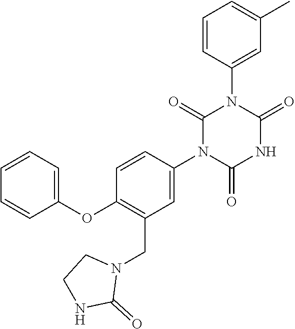 [SMILES: Cc1cccc(-n2c(=O)[nH]c(=O)n(-c3ccc(Oc4ccccc4)c(CN4CCNC4=O)c3)c2=O)c1]

To a microwave vial previously equipped with a magnetic stirrer and nitrogen balloon was added 2-Imidazolidinone (0.039 g, 0.458 mmol) in THF (1.0 ml) and the mixture was cooled to 0° C. Sodium hydride (60%, 0.018 g, 0.458 mmol) was added at 0° C. and reaction mixture was stirred for 20 min at 0° C. 1-[3-(chloromethyl)-4-phenoxyphenyl]-3-(3-methylphenyl)-1,3,5-triazinane-2,4,6-trione (Intermediate 11, 0.100 g, 0.229 mmol) was added and resulting reaction mixture was stirred room temperature for 12 h. The completion of reaction was confirmed by the TLC using DCM:MeOH (9:1) as mobile phase. The TLC was visualized using UV light. After completion of the reaction, reaction mixture was quenched with water (10 ml) and product was extracted with EtOAc (3×15 ml). The combined organic layer was washed with brine (35 ml), dried over sodium sulphate and the solvent removed under reduced. The crude product was purified by preparative HPLC (acetonitrile 20-100% in water [5 mM Ammonium bicarbonate+0.1% NH₃]) to yield 0.006 g (5% yield) of the title compound. ¹H NMR (400 MHz, DMSO-d₆): δ 11.96 (s, 1H), 7.44-7.42 (m, 2H), 7.40-7.35 (m, 2H), 7.26-7.16 (m, 5H), 7.08-6.98 (m, 2H), 6.90 (d, J=8.8 Hz, 1H), 6.47 (s, 1H), 4.28 (s, 2H), 3.25-3.26 (m, 4H), 2.33 (s, 3H); MS (ES+) m/z 486 [M+H]⁺.

### Example 6

### 1-(3-ethyl-4-phenoxyphenyl)-3-phenyl-1,3,5-triazinane-2,4,6-trione

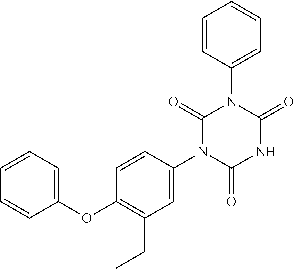 [SMILES: CCc1cc(-n2c(=O)[nH]c(=O)n(-c3ccccc3)c2=O)ccc1Oc1ccccc1]

In a microwave vial previously equipped with a magnetic stirrer and nitrogen balloon was taken 3-(3-ethyl-4-phenoxyphenyl)-1-phenylurea (Intermediate 15, 1.20 g, 3.6 mmol) in Bromobenzene (14.0 ml). Ethoxy carbonyl isocyanate (1.246 g, 10 mmol) was added and the resulting reaction mixture heated at 150° C. for 4 h in an Anton paar microwave synthesizer-300. The solvent was removed under reduced pressure to obtain crude product that was purified by preparative RP-HPLC (acetonitrile 55-100% in water [0.1% formic acid]) to yield 130 mg (8.9% yield) of the title compound. ¹H NMR (400 MHz, DMSO-d₆): δ 12.05 (s, 1H), 7.51-7.33 (m, 8H), 7.21 (d, J=7.6 Hz, 1H), 7.13 (t, J=7.2 Hz, 1H), 7.01-6.95 (m, 2H), 6.90 (d, J=8.4 Hz, 1H), 2.60 (q, J=7.20 Hz, 2H), 1.15 (t, J=7.60 Hz, 3H). MS (ES-) m/z 400 [M−H]⁻.

### Example 7

### 1-(3-ethyl-4-phenoxyphenyl)-3-(3-methylphenyl)-1,3,5-triazinane-2,4,6-trione

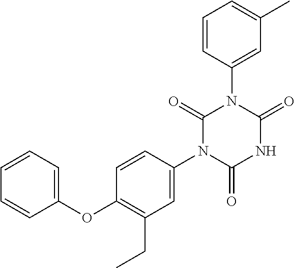 [SMILES: CCc1cc(-n2c(=O)[nH]c(=O)n(-c3cccc(C)c3)c2=O)ccc1Oc1ccccc1]

In a 30 ml microwave vial previously equipped with a magnetic stirrer and nitrogen balloon was taken 3-(3-ethyl-4-phenoxyphenyl)-1-(3-methylphenyl)urea (Intermediate 16, 1.20 g, 3.4 mmol) in Bromobenzene (12 ml). Ethoxy carbonyl isocyanate (1.19 g, 10.4 mmol) was added and the resulting reaction mixture was heated to 150° C. for 4 h in an Anton paar microwave synthesizer-300. The solvent was removed under reduced pressure to obtain crude product that was purified by preparative RP-HPLC (acetonitrile 60-100% in water [0.1% formic acid]) to yield 84 mg (5%) of the title compound. 1H NMR (400 MHz, DMSO-d6): δ 11.99 (s, 1H), 7.43-7.32 (m, 4H), 7.26-7.11 (m, 5H), 7.01-6.95 (m, 2H), 6.90 (d, J=8.4 Hz, 1H), 2.60 (m, 2H), 2.34 (s, 3H) 1.16 (t, J=7.20 Hz, 3H). MS (ES-) m/z 416 [M−H]⁻.

### Example 8

### 1-(4-methoxyphenyl)-3-(3-methyl-4-phenoxyphenyl)-1,3,5-triazinane-2,4,6-trione

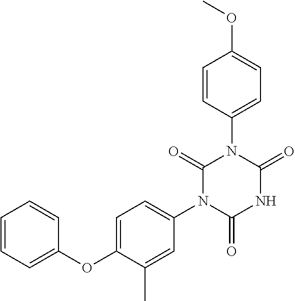 [SMILES: COc1ccc(-n2c(=O)[nH]c(=O)n(-c3ccc(Oc4ccccc4)c(C)c3)c2=O)cc1]

In a RBF previously equipped with a magnetic stirrer and nitrogen balloon was taken 1-(4-methoxyphenyl)-3-[1-(4-methoxyphenyl)-5-(3-methyl-4-phenoxyphenyl)-4-(methylsulfanyl)-6-oxo-1,2,5,6-tetrahydro-1,3,5-triazin-2-ylidene]urea (Intermediate 18, 0.45 g, 0.7 mmol) in 1,4-Dioxane (4.5 ml). To the solution 2M aq. HCl (6.75 ml) was added and the reaction mixture was heated to 100° C. and stirred at same temperature for 2 h. The reaction mixture was allowed to reach room temperature and quenched with ice cold water (20 ml) and stirred for 10 min. The aqueous layer was extracted with Ethyl Acetate (2×30 ml) and the combined organic layer was washed with ice cold water (20 ml) followed by brine (20 ml), dried over sodium sulphate and the solvent removed under reduce pressure to obtain crude product. The crude product was purified by column chromatography using 25% ethyl acetate in hexanes as an eluent to yield 0.085 g (13%) of the title compound. ¹H NMR (400 MHz, DMSO-d₆: δ 11.96 (s, 1H), 7.46-7.39 (m, 2H), 7.33-7.28 (m, 3H), 7.22 (dd, J=8.4 Hz, 2.0 Hz, 1H), 7.15 (t, J=7.2 Hz, 1H), 7.03-6.97 (m, 4H), 6.92 (d, J=8.8 Hz, 1H), 3.80 (s, 3H), 2.23 (s, 3H). MS (ES+) m/z 418 [M+H]⁺.

### Example 9

### 1-(3-methylphenyl)-3-{3-[2-oxo-2-(pyrrolidin-1-yl)ethoxy]-4-phenoxyphenyl}-1,3,5-triazinane-2,4,6-trione

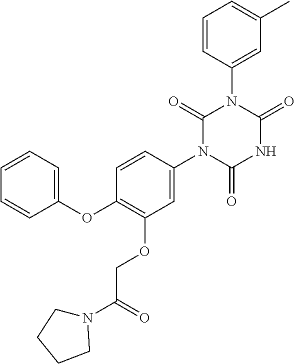 [SMILES: Cc1cccc(-n2c(=O)[nH]c(=O)n(-c3ccc(Oc4ccccc4)c(OCC(=O)N4CCCC4)c3)c2=O)c1]

In a vial previously equipped with a magnetic stirrer and nitrogen balloon was taken 2-{5-[3-(3-methylphenyl)-2,4,6-trioxo-1,3,5-triazinan-1-yl]-2-phenoxyphenoxy}acetic acid (Intermediate 22, 0.100 g, 0.216 mmol) in DMF (1.0 ml). To it HATU (0.247 g, 0.650 mmol) was added and resulting reaction mixture was stirred for 1 h at 0° C. After that, pyrrolidine (0.023 g, 0.325 mmol) and N,N-Diisopropylethylamine (0.15 ml, 0.866 mmol) were added and the reaction mixture was allowed to reach room temperature and then stirred for 2 h. The reaction mixture was diluted with ice cold water (100 ml) and extracted with EtOAc (3×50 ml). The combined organic layer was washed with brine (100 ml), dried over sodium sulphate and the solvent removed under reduced pressure to obtain crude product that was purified using preparative RP-HPLC (acetonitrile 10-100% in water [5 mM ammonium bicarbonate+0.1% NH₃]) to yield 42 mg (37% yield) of the title compound. ¹H NMR (400 MHz, DMSO-d₆): δ 11.85 (s, 1H), 7.39-7.31 (m, 3H), 7.23 (d, J=7.2 Hz, 1H), 7.16 (app s, 3H), 7.11-7.04 (m, 2H), 7.02-6.92 (m, 3H), 4.67 (s, 2H), 3.32-3.23 (m, 4H), 2.34 (s, 3H), 1.85-1.67 (m, 4H). MS (ES-) m/z 513 [M−H]⁻.

### Example 10

### 1-(3-methylphenyl)-3-{3-[2-oxo-2-(piperidin-1-yl)ethoxy]-4-phenoxyphenyl}-1,3,5-triazinane-2,4,6-trione

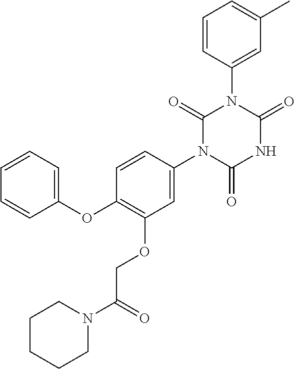 [SMILES: Cc1cccc(-n2c(=O)[nH]c(=O)n(-c3ccc(Oc4ccccc4)c(OCC(=O)N4CCCCC4)c3)c2=O)c1]

In a vial previously equipped with a magnetic stirrer and nitrogen balloon was taken 2-{5-[3-(3-methylphenyl)-2,4,6-trioxo-1,3,5-triazinan-1-yl]-2-phenoxyphenoxy}acetic acid (Intermediate 22, 0.100 g, 0.216 mmol) in DMF (1.0 ml). To it HATU (0.247 g, 0.650 mmol) was added and resulting reaction mixture was stirred for 1 h at 0° C. After that, piperidine (0.027 g, 0.325 mmol) and N,N-Diisopropylethylamine (0.15 ml, 0.866 mmol) were added and the reaction mixture was allowed to reach room temperature and then stirred for 2 h at room temperature. The reaction mixture was diluted with ice cold water (100 ml) and extracted with EtOAc (3×50 ml). The combined organic layer was washed with brine (100 ml), dried over sodium sulphate and the solvent removed under reduced pressure to obtain crude product that was purified by preparative RP-HPLC (acetonitrile 20-100% in water [5 mM ammonium bicarbonate+0.1% N H₃]) to yield 30 mg (26% yield) of the title compound. ¹H NMR (400 MHz, DMSO-d₆): δ 11.58 (s, 1H), 7.38-7.32 (m, 3H), 7.23 (d, J=7.2 Hz, 1H), 7.18-7.12 (m, 3H), 7.10-7.04 (m, 2H), 7.01-6.91 (m, 3H), 4.75 (s, 2H), 3.42-3.35 (m, 2H), 3.26 (br s, 2H), 2.34 (s, 3H), 1.51 (br s, 2H), 1.40 (br s, 4H). MS (ES-) m/z 527 [M−H]⁻.

### Example 11

### 1-(3-methylphenyl)-3-{3-[2-(morpholin-4-yl)-2-oxoethoxy]-4-phenoxyphenyl}-1,3,5-triazinane-2,4,6-trione

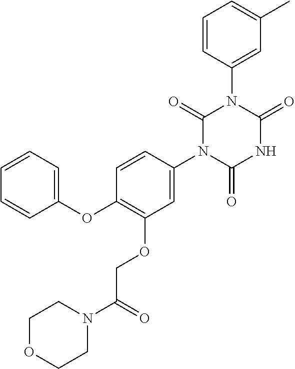 [SMILES: Cc1cccc(-n2c(=O)[nH]c(=O)n(-c3ccc(Oc4ccccc4)c(OCC(=O)N4CCOCC4)c3)c2=O)c1]

In a vial previously equipped with a magnetic stirrer and nitrogen balloon was taken 2-{5-[3-(3-methylphenyl)-2,4,6-trioxo-1,3,5-triazinan-1-yl]-2-phenoxyphenoxy}acetic acid (Intermediate 22, 0.100 g, 0.216 mmol) in DMF (1.0 ml). To it HATU (0.247 g, 0.650 mmol) was added and resulting reaction mixture was stirred for 1 h at 0° C. After that, morpholine (0.028 g, 0.325 mmol) and N,N-Diisopropylethylamine (0.15 ml, 0.866 mmol) were added and the reaction mixture was allowed to reach room temperature and then stirred for 2 h at room temperature. The reaction mixture was diluted with ice cold water (100 ml) and extracted with EtOAc (3×50 ml). The combined organic layer was washed with brine (100 ml), dried over sodium sulphate and the solvent removed under reduced pressure to obtain crude product that was purified by preparative RP-HPLC (acetonitrile 10-100% in water [5 mM ammonium bicarbonate+0.1% N H₃]) to yield 17 mg (14% yield) of the title compound. ¹H NMR (400 MHz, DMSO-d₆): δ 11.99 (s, 1H), 7.42-7.30 (m, 3H), 7.24 (d, J=8 Hz, 1H), 7.22-7.13 (m, 3H), 7.12-7.03 (m, 2H), 7.01 (d, J=7.2 Hz, 1H), 6.98-6.89 (d, 2H), 4.79 (s, 2H), 3.56-3.45 (m, 4H), 3.45-3.36 (m, 4H), 2.34 (s, 3H). MS (ES-) m/z 529 [M−H]⁻.

### Example 12

### 1-{3-[2-(azetidin-1-yl)-2-oxoethoxy]-4-phenoxyphenyl}-3-(3-methylphenyl)-1,3,5-triazinane-2,4,6-trione

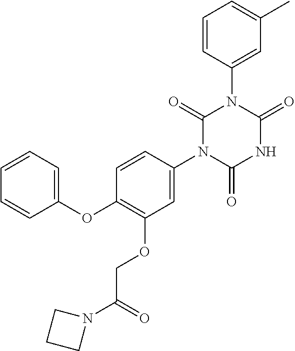 [SMILES: Cc1cccc(-n2c(=O)[nH]c(=O)n(-c3ccc(Oc4ccccc4)c(OCC(=O)N4CCC4)c3)c2=O)c1]

In a vial previously equipped with a magnetic stirrer and nitrogen balloon was taken 2-{5-[3-(3-methylphenyl)-2,4,6-trioxo-1,3,5-triazinan-1-yl]-2-phenoxyphenoxy}acetic acid (Intermediate 22, 0.075 g, 0.162 mmol) in DMF (0.75 ml). To it HATU (0.185 g, 0.487 mmol) was added and the resulting mixture was stirred for 1 h at 0° C. After that, azetidine (0.014 g, 0.243 mmol) and N,N-Diisopropylethylamine (0.11 ml, 0.650 mmol) were added and the reaction mixture was allowed to reach room temperature and then stirred for 2 h at room temperature. The reaction mixture was diluted with ice cold water (100 ml) and extracted with EtOAc (3×50 ml). The combined organic layer was washed with brine (100 ml), dried over sodium sulphate and the solvent removed under reduced pressure to obtain crude product that was purified by preparative RP-HPLC (acetonitrile 40-100% in water [0.1% formic acid]) to yield 9 mg (11% yield) of the title compound. ¹H NMR (400 MHz, DMSO-d₆): δ 12.02 (s, 1H), 7.41-7.32 (m, 3H), 7.23 (d, J=7.6 Hz, 1H), 7.16 (app s, 3H), 7.12-7.05 (m, 2H), 7.04-6.99 (m, 1H), 6.98-6.92 (m, 2H), 4.53 (s, 2H), 4.07 (t, J=7.2 Hz, 2H), 3.85 (t, J=7.6 Hz, 2H), 2.34 (s, 3H), 2.18-2.10 (m, 2H). MS (ES-) m/z 499 [M−H]⁻.

### Example 13

### 1-[3-(methylsulfanyl)-4-phenoxyphenyl]-3-phenyl-1,3,5-triazinane-2,4,6-trione

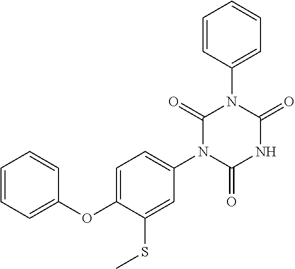 [SMILES: CSc1cc(-n2c(=O)[nH]c(=O)n(-c3ccccc3)c2=O)ccc1Oc1ccccc1]

In a microwave vial previously equipped with a magnetic stirrer and nitrogen balloon was taken 3-[3-(methylsulfanyl)-4-phenoxyphenyl]-1-phenylurea (Intermediate 25, 0.150 g, 0.428 mmol) in Chlorobenzene (1.5 ml). The solution was cooled to 0° C. and Ethoxy carbonyl isocyanate (0.197 g, 1.712 mmol) was added and resulting reaction mixture heated to 150° C. for 4 h in an Anton paar microwave synthesizer-300. After completion of the reaction, solvent was evaporated under reduced pressure to obtain crude product. The crude product was purified by preparative RP-HPLC (acetonitrile 25-100% in water [0.1% formic acid]) to yield 0.023 g (12% yield) of the title compound. ¹H NMR (400 MHz, DMSO-d₆: δ 12.07 (s, 1H), 7.54-7.37 (m, 8H), 7.21-7.14 (m, 2H), 7.03-6.94 (m, 3H), 2.43 (s, 3H). MS (ES-) m/z 418 [M−H]⁻.

### Example 14

### 1-(3-methylphenyl)-3-[3-(methylsulfanyl)-4-phenoxyphenyl]-1,3,5-triazinane-2,4,6-trione

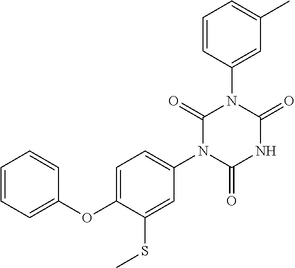 [SMILES: CSc1cc(-n2c(=O)[nH]c(=O)n(-c3cccc(C)c3)c2=O)ccc1Oc1ccccc1]

In a microwave tube previously equipped with a magnetic stirrer and nitrogen balloon was taken 1-(3-methylphenyl)-3-[3-(methylsulfanyl)-4-phenoxyphenyl]urea (Intermediate 26, 0.150 g, 0.411 mmol) in Toluene (1.5 ml). The solution was cooled to 0° C. and Ethoxy carbonyl isocyanate (0.189 g, 1.646 mmol) was added and resulting reaction mixture was allowed to reach room temperature and then heated at 150° C. for 3 h in an Anton paar microwave synthesizer-300. After completion of the reaction, solvent was evaporated under reduced pressure to obtain the crude product that was purified by preparative RP-HPLC (acetonitrile 50-100% in water [0.1% formic acid]) to yield 0.011 g (6% yield) of the title compound. ¹H NMR (400 MHz, DMSO-d₆: δ 12.05 (s, 1H), 7.44-7.32 (m, 4H), 7.24 (d, J=7.20 Hz, 1H), 7.19-7.12 (m, 4H), 7.01-6.92 (m, 3H), 2.41 (s, 3H), 2.34 (s, 3H). MS (ES-) m/z 432 [M−H]⁻.

### Example 15

### 1-(3-methylphenyl)-3-{3-[(2-oxopyrrolidin-1-yl)methyl]-4-phenoxyphenyl}-1,3,5-triazinane-2,4,6-trione

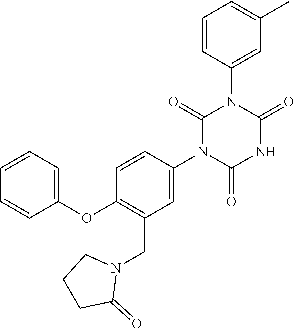 [SMILES: Cc1cccc(-n2c(=O)[nH]c(=O)n(-c3ccc(Oc4ccccc4)c(CN4CCCC4=O)c3)c2=O)c1]

In a sealed tube previously equipped with a magnetic stirrer and nitrogen balloon was taken 1-[3-(chloromethyl)-4-phenoxyphenyl]-3-(3-methylphenyl)-1,3,5-triazinane-2,4,6-trione (Intermediate 11, 0.10 g, 0.2 mmol) in THF (1.0 ml) and to it NaH (60%, 0.018 g, 0.4 mmol) was added and the mixture was stirred for 10 min at room temperature. 2-Pyrrolidone (0.039 g, 0.4 mmol) was added and the reaction mixture was heated at 60° C. for 1 h. The reaction mixture was quenched with water (10 ml) and the aqueous layer was extracted with ethyl acetate (3×10 ml). The combined organic layer was dried over sodium sulphate and the solvent removed under reduced pressure to obtain crude product that was purified by preparative RP-HPLC (acetonitrile 15-100% in water [5 mM Ammonium bicarbonate+0.1% NH₃]) to yield 15 mg (13% yield) of the title compound. ¹H NMR (400 MHz, DMSO-d₆): δ 11.97 (s, 1H), 7.43-7.17 (m, 9H), 7.03-6.92 (m, 3H), 4.42 (s, 2H), 3.27 (br s, 2H), 2.35 (s, 3H), 2.22 (br s, 2H), 1.90 (br s, 2H). MS (ES-) m/z 483 [M−H]⁻.

### BIOLOGICAL EXAMPLES

### Biological Example 1

### In Vitro Trk Receptor Modulation Assay

A high throughput cell-based screen was used to identify positive modulators of TrkA and TrkB. The screen involves the use of cell-based assay overexpressing TrkA, or TrkB. The purpose of the assay is to identify compounds that modulate neurotrophin signalling (Forsell et al 2012). The assay can be used in inhibitor mode using a high concentration of ligand, in modulator mode using an intermediate concentration and in agonist mode using a low concentration of ligand.

The assay uses Enzyme Fragment Complementation (EFC) technique, which is a proximity-based assay. Briefly, cells used in this assay over-express two fusion proteins, i.e. the receptor, which can be one of TrkA or TrkB fused to a small peptide of beta-galactosidase and an adaptor protein, i.e. SHC1 (or any other Trk-adaptor protein) fused to the major part of beta-galactosidase. Ligand binding to the receptor induces phosphorylation of the intracellular domain and hence, recruitment of the adaptor protein to the receptor. The proximity between the small activating peptide on the receptor and the major part of beta-galactosidase on the adaptor protein leads to an active beta-galactosidase enzyme. The activation of the receptor is quantified by measuring the amount of active beta-galactosidase by its conversion of a non-luminescent substrate into a luminescent product.

U2OS-cells, over-expressing TrkA or TrkB, were plated in 96- or 384-well plates and incubated overnight. On the following day, test compound was pre-mixed with ligand (NGF) and the ligand-compound mixture is then added to the cells to yield a final ligand concentration of 10 ng/mL. After 3 hours of incubation at room temperature, the incubation is stopped by the addition of a beta-galactosidase substrate mixture containing detergents. The substrate mixture is incubated for 60 minutes at ambient temperature. The luminescence is thereafter read by the use of a plate reader.

### Results

TABLE 1
Data from these assays for representative compounds is shown in the
table below. The potency is expressed as EC50 (μM) for the individual
receptors. The data indicate that the compounds of the invention are
highly potent modulators of TrkA and TrkB receptor signalling and
are therefore expected to possess useful therapeutic properties.

| Example | TrkA | TrkB |
|---|---|---|
| 1 | 0.12 | 0.15 |
| 2 | 0.43 | 0.26 |
| 3 | 0.33 | 0.31 |
| 4 | 0.30 | 0.27 |
| 5 | 0.30 | 0.12 |
| 6 | 0.21 | 0.22 |
| 7 | 0.13 | 0.09 |
| 8 | 0.35 | 0.23 |
| 9 | 0.21 | 0.15 |
| 10 | 0.28 | 0.32 |
| 11 | 0.27 | 0.18 |
| 12 | 0.41 | 0.22 |
| 13 | 0.64 | 0.38 |
| 14 | 0.65 | 0.30 |
| 15 | 0.04 | 0.11 |

### Biological Example 2

### Human Liver Microsomal Stability

A 10 mM stock solution (in DMSO) was prepared for each compound. From the intermediate stock solution of 2 mM, a working solution of 0.5 mM was prepared by diluting the compound in Acetonitrile:water (50:50). The compound (1.8 μL of working solution) was spiked in 0.1 M potassium phosphate buffer (260.7 μL), pH 7.4 at a concentration of 3 μM (0.1% DMSO). Following this, human liver microsomes (pooled, Invitrogen, #HMMCPL) (7.5 μL; final protein conc. 0.5 mg/mL) were added. The aforementioned sample was incubated at 37° C. for 5 min. Subsequently, 30 μL of 10 mM NADPH prepared in 0.1 M potassium phosphate buffer was added (as a co-factor) to initiate the reaction. The samples were then incubated at 37° C. for 0, 20, 40, 60 and 120 min.

At each time point (0, 20, 40, 60 and 120 min), 40 μL of the samples were withdrawn and reactions were stopped using 360 μL chilled acetonitrile or methanol containing suitable internal standard (Carbamazepine). The samples were centrifuged and the supernatants was analyzed in duplicate by LC-MS/MS. The percent compound remaining at each time point was calculated with respect to that of the 0 min sample. Control samples were run without NADPH for initial and final time point and blank samples was prepared using DMSO (without the test compound).

For comparison, the experiment was also performed with known Trk receptor signalling modulator 1-methyl-3-[3-methyl-4-(4-trifluoromethanesulfonylphenoxy)phenyl]-1,3,5-triazinane-2,4,6-trione) (ponazuril). Ponazuril is metabolized very slowly in vivo and this prolonged exposure creates an increased risk of side effects if the compound is administered systemically. This causes significant challenges to the development of the compound as a systemic drug.

### Results

TABLE 2
% metabolism of representative compounds in human
liver microsomes after incubation for 120 min.

| | % of parent compound metabolized |
|---|---|
| Example | after 120 min |
| 1 | 26.8 |
| 2 | 13.3 |
| 3 | 82.0 |
| 4 | 37.7 |
| 5 | 15.4 |
| 6 | NT |
| 7 | 48.8 |
| 8 | NT |
| 9 | 82.8 |
| 10 | 99.8 |
| 11 | 57.3 |
| 12 | NT |
| 13 | 4.7 |
| 14 | 53.4 |
| 15 | 29.6 |
| ponazuril | 3.1 |

These data indicate that the compounds are metabolised more quickly than ponazuril and are therefore likely to have improved metabolic profiles for pharmaceutical development as systemic drugs and/or may offer other metabolic advantages for pharmaceutical development.

### Biological Example 3

### Passive Avoidance Task

The passive avoidance (PA) is an aversive learning task based on classical (Pavlovian) fear conditioning that allows for analysis of both facilitation and impairment of memory function by adjusting the unconditioned stimulus, i.e. the electrical foot shock. Commonly a cognitive-impairing agent is administered to the animals to mimic the neurochemical disturbances present in various cognitive disorders e.g. cholinergic (scopolamine) and glutamatergic (MK-801) deficits.

Prior to testing, the animals are brought to the experimental room where they were allowed to habituate for 60 min. The test is conducted using a modified shuttle box with two communicating compartments of equal size with a small sliding door built into the separating wall and a stainless-steel bar floor. One of the compartments is not illuminated and thus black whereas the other compartment (the light one) is illuminated by an electric bulb, installed on the top of a plexiglass cover. The PA training is conducted in a single session. The animals are allowed to explore the compartment for 60 sec, after which the sliding door is automatically opened and the mouse is allowed to cross over into the dark compartment. Once the mouse has entered the dark chamber with all four feet, the sliding door is automatically closed and a scrambled electrical current is delivered through the grid floor. Latency to cross over into the dark compartment (training latency) is recorded. The memory test is conducted on day 2 (24 hrs later) whereby the animals are placed in the light compartment and allowed to explore for 15 sec, then the sliding door is opened allowing free access to the dark compartment for a period of 300 sec. The latency to cross over into the dark compartment with all four feet is measured (retention latency) as well as time in bright compartment and a number of other relevant parameters (e.g. number of visits in the dark compartment).

Vehicle (20% DMSO in 0.1M PBS) or different doses of the compounds of Example 1 and Example 3 were administered to C57/Bl6 mice as a single s.c. administration 60 min prior to PA training. PA training was then performed according to the procedure described above. On the day of PA training, scopolamine at 0.3 mg/kg, or vehicle, was administered subcutaneously 30 min prior to training.

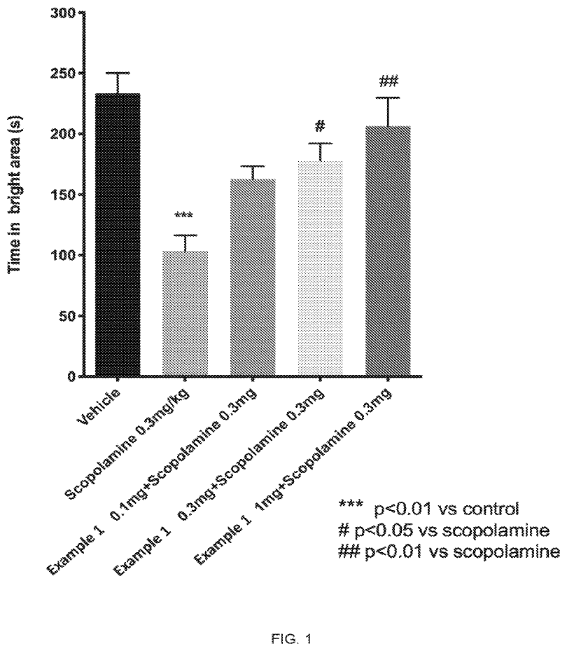

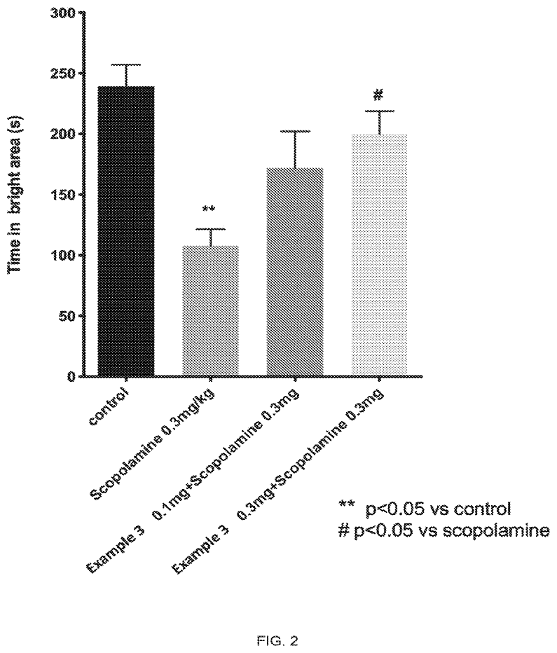

Data on time spend in the bright compartment for mice treated with different doses of the compound of Example 1, the vehicle control group and scopolamine alone are shown in FIG. 1, and for mice treated with different doses of the compound of Example 3, the vehicle control group and scopolamine alone are shown in FIG. 2.

### Biological Example 4

### Forced Swim Test

The Forced Swim Test (FST) is a commonly used test in rodents to assess depression-like behaviour (Porsolt et al., Nature, 1977, 266, 730-732). The model measures a learned helplessness response and has shown to have predictive validity for clinical antidepressants, e.g. SSRIs.

The mouse version consists of a vertical glass cylinder (25 cm high, 13 cm in diameter) filled with tap water up to 16 cm in height (25±0.5° C.). This means that the animal cannot escape (climb out) nor stand on its tail or hindlimbs. After a certain time the animal realize that it cannot get out and will stop struggling to get out, and instead become immobile and float.

The animal is placed inside the cylinder and allowed two swimming sessions: a 10 min pre-test (on day 1), 24 h later (day 2) a second 6 min test is conducted. The total duration of immobility and latency to the first immobility are recorded during the second 6 min test. Immobility is defined as floating passively in an upright position in the water, with only small movements necessary to keep the head above the water surface. Animals are then gently removed from water and dried.

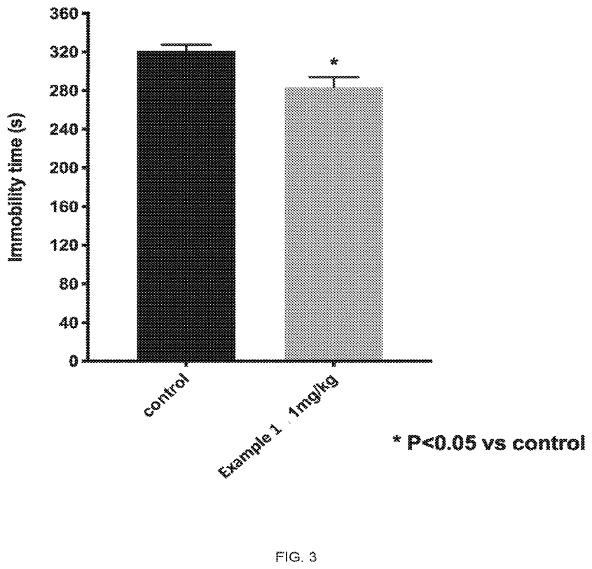

Vehicle (20% DMSO in 0.1M PBS) or 1 mg/kg of the compound of Example 1 were administered to C57/B16 mice as a single s.c. administration 30 min prior to FST on day 2. As comparator, fluoxetine is commonly used a dose of 20 mg/kg. Data on the total time of immobility for mice treated with the compound of Example 1 or the vehicle control are shown in FIG. 3.

1-26. (canceled)

27. A compound of formula I, 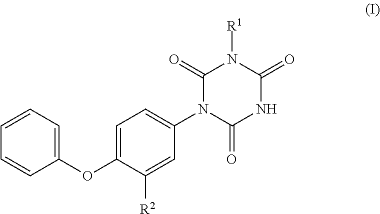
wherein:
R¹ represents 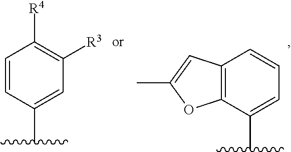
wherein 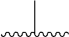
represents the point of attachment to the nitrogen atom;
R³ represents hydrogen, methyl or methoxymethyl;
R⁴ represents hydrogen, methoxy or methoxymethyl;
R² represents methyl, ethyl, methoxymethyl, methylsulfanyl or -A-R⁵; wherein
A represents C₁₋₂ alkylene, —C₁₋₂alkyleneO-, or —OC₁₋₂alkylene-, which three groups are optionally substituted by one or more groups selected from halo, C₁₋₂ alkyl and ═0; and
R⁵ represents oxetanyl or a 4-7-membered nitrogen-containing heterocyclyl group, each of which groups are optionally substituted by one or more groups selected from halo, C₁₋₂ alkyl and ═O;
or a pharmaceutically-acceptable salt thereof, with the proviso that the compound of formula I does not represent

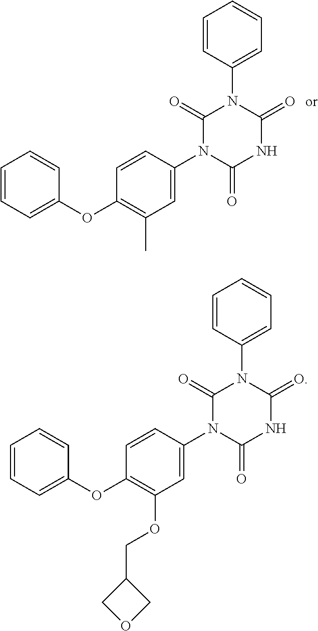 [SMILES: Cc1cc(-n2c(=O)[nH]c(=O)n(-c3ccccc3)c2=O)ccc1Oc1ccccc1 ; O=c1[nH]c(=O)n(-c2ccc(Oc3ccccc3)c(OCC3COC3)c2)c(=O)n1-c1ccccc1]

28. A compound as claimed in claim 27, wherein R¹ represents 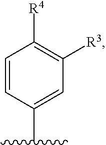
wherein R³ and R⁴ are as defined in claim 1.

29. A compound as claimed in claim 27, wherein at least one of R³ and R⁴ represents hydrogen.

30. A compound as claimed in claim 27, wherein R³ represents methyl or methoxymethyl and R⁴ represents hydrogen.

31. A compound as claimed in claim 27, wherein R³ represents hydrogen and R⁴ represents methoxy.

32. A compound as claimed in claim 27, wherein R³ and R⁴ each represent hydrogen.

33. A compound as claimed in claim 27, wherein R² represents methyl, ethyl or methoxymethyl.

34. A compound as claimed in claim 27, wherein R² represents ethyl or methoxymethyl.

35. A compound as claimed in claim 27, wherein R² represents -A-R⁵, wherein A and R⁵ are as defined in claim 27.

36. A compound as claimed in claim 35, wherein R⁵ represents a 4-7-membered nitrogen-containing heterocyclyl group, which heterocyclyl group is optionally substituted by one or more groups selected from halo, C₁₋₂ alkyl and ═O.

37. A compound as claimed in claim 27, wherein A represents —CH₂— or —OCH₂C(O)—.

38. A compound as claimed in claim 27, wherein R⁵ represents a 4-6-membered nitrogen-containing heterocyclyl group, which heterocyclyl group is optionally substituted by one or more groups selected from halo, C₁₋₂ alkyl and ═O.

39. A compound as claimed in claim 38, wherein R⁵ represents a 4-6-membered nitrogen-containing heterocyclyl group selected from: 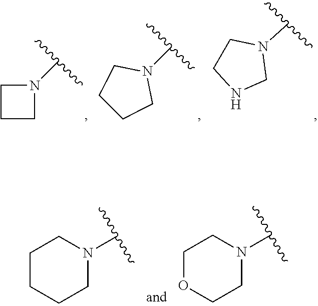 [SMILES: *N1CCC1 ; *N1CCCC1 ; *N1CCNC1 ; *N1CCCCC1 ; *N1CCOCC1]
wherein 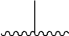
represents the point of attachment to A, each of which heterocyclyl groups is optionally substituted by a ═O group.

40. A compound as claimed in claim 27, wherein the compound is selected from the group consisting of: 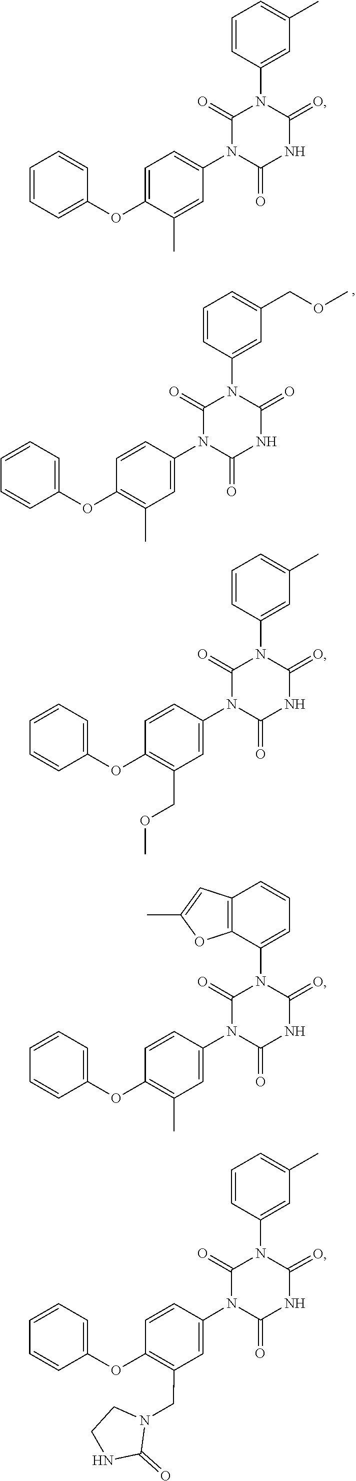 [SMILES: Cc1cccc(-n2c(=O)[nH]c(=O)n(-c3ccc(Oc4ccccc4)c(C)c3)c2=O)c1 ; COCc1cccc(-n2c(=O)[nH]c(=O)n(-c3ccc(Oc4ccccc4)c(C)c3)c2=O)c1 ; COCc1cc(-n2c(=O)[nH]c(=O)n(-c3cccc(C)c3)c2=O)ccc1Oc1ccccc1 ; Cc1cc2cccc(-n3c(=O)[nH]c(=O)n(-c4ccc(Oc5ccccc5)c(C)c4)c3=O)c2o1 ; Cc1cccc(-n2c(=O)[nH]c(=O)n(-c3ccc(Oc4ccccc4)c(CN4CCNC4=O)c3)c2=O)c1] 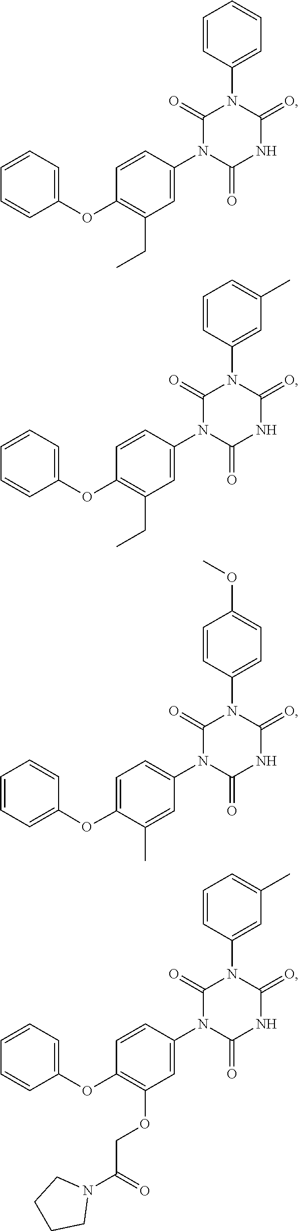 [SMILES: CCc1cc(-n2c(=O)[nH]c(=O)n(-c3ccccc3)c2=O)ccc1Oc1ccccc1 ; CCc1cc(-n2c(=O)[nH]c(=O)n(-c3cccc(C)c3)c2=O)ccc1Oc1ccccc1 ; COc1ccc(-n2c(=O)[nH]c(=O)n(-c3ccc(Oc4ccccc4)c(C)c3)c2=O)cc1 ; Cc1cccc(-n2c(=O)[nH]c(=O)n(-c3ccc(Oc4ccccc4)c(OCC(=O)N4CCCC4)c3)c2=O)c1] 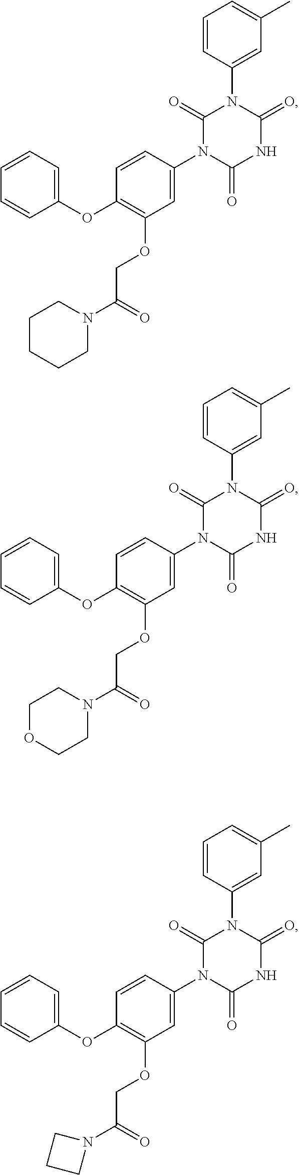 [SMILES: Cc1cccc(-n2c(=O)[nH]c(=O)n(-c3ccc(Oc4ccccc4)c(OCC(=O)N4CCCCC4)c3)c2=O)c1 ; Cc1cccc(-n2c(=O)[nH]c(=O)n(-c3ccc(Oc4ccccc4)c(OCC(=O)N4CCOCC4)c3)c2=O)c1 ; Cc1cccc(-n2c(=O)[nH]c(=O)n(-c3ccc(Oc4ccccc4)c(OCC(=O)N4CCC4)c3)c2=O)c1] 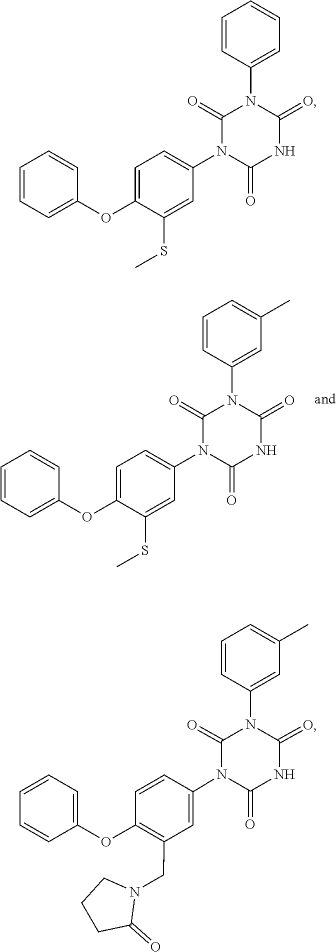 [SMILES: CSc1cc(-n2c(=O)[nH]c(=O)n(-c3ccccc3)c2=O)ccc1Oc1ccccc1 ; CSc1cc(-n2c(=O)[nH]c(=O)n(-c3cccc(C)c3)c2=O)ccc1Oc1ccccc1 ; Cc1cccc(-n2c(=O)[nH]c(=O)n(-c3ccc(Oc4ccccc4)c(CN4CCCC4=O)c3)c2=O)c1]
or a pharmaceutically-acceptable salt thereof.

41. A pharmaceutical composition comprising a compound as defined in claim 27, or a pharmaceutically acceptable salt thereof, in combination with one or more pharmaceutically-acceptable excipients.

42. A method of treating a disease characterised by impaired signalling of neurotrophins and/or other trophic factors, which comprises administering to a patient in need thereof a therapeutically effective amount of a compound as defined in claim 27, or a pharmaceutically acceptable salt thereof.

43. The method of claim 42, wherein the disease characterised by impaired signalling of neurotrophins and/or other trophic factors is selected from Alzheimer's disease, Lewy body dementia, frontotemporal dementia, cognitive dysfunction, mild cognitive impairment, other dementia disorders, Parkinson's, disease, other Parkinsonian disorders and/or other tauopathies, Huntington's disease, brain injuries, stroke, motor neurone diseases, multiple sclerosis, spinal cord injury, hypoxia, ischemia, hypoxic ischemia injury, coronary artery disease, obesity, diabetes, metabolic syndrome, diabetic neuropathy, Charcot-Marie-Tooth disease and its variants, nerve injury, genetic or acquired or traumatic hearing loss, blindness, posterior eye diseases, anterior eye diseases, dry eye syndrome, neurotrophic keratitis, glaucoma, high intraocular pressure, retinitis pigmentosa, post-traumatic stress disorders, WAGR syndrome, Prader-Willi syndrome, olfactory decline, olfactory dysfunction, fragile X syndrome, congenital hypoventilation syndrome, obsessive-compulsive disorder, anxiety, generalised anxiety disorder, schizophrenia, depression, eating disorders, bipolar disorder, chronic fatigue syndrome, neuromyelitis optica, Rett syndrome, epilepsy, Friedreich's ataxia, obstructive sleep apnea-hypopnea syndrome, pain and constipation.

44. The method of claim 42, wherein the disease characterised by impaired signalling of neurotrophins and/or other trophic factors is selected from the group consisting of Alzheimer's disease, Parkinson's disease, other Parkinsonian diseases, other tauopathies, Lewy body dementia, motor neurone disease, Pick's disease, obesity, metabolic syndrome, diabetes, diabetic neuropathy, glaucoma, dry eye syndrome, neurotrophic keratitis, genetic, acquired or traumatic hearing loss and Rett Syndrome.

45. The method of claim 42, wherein the disease characterised by impaired signalling of neurotrophins and/or other trophic factors is selected from the group consisting of Alzheimer's disease, Parkinson's disease, cognitive dysfunction, depression, diabetic neuropathy, glaucoma, dry eye syndrome, genetic, acquired or traumatic hearing loss and Rett syndrome.

46. The method of claim 42, wherein the disease characterised by impaired signalling of neurotrophins and/or other trophic factors is Alzheimer's disease.

47. A combination product comprising:
(I) a compound as defined in claim 27, or a pharmaceutically acceptable salt thereof; and
(II) one or more other therapeutic agent that is useful in the treatment of a disease characterised by impaired signalling of neurotrophins and/or other trophic factors, wherein each of components (I) and (II) is formulated in admixture, optionally with a pharmaceutically-acceptable excipient.

48. A kit-of-parts comprising:
(a) a pharmaceutical composition comprising a compound as defined in claim 27, or a pharmaceutically acceptable salt thereof, formulated in admixture with a pharmaceutically-acceptable excipient; and
(b) a pharmaceutical composition comprising one or more other therapeutic agent that is useful in the treatment of a disease characterised by impaired signalling of neurotrophins and/or other trophic factors, formulated in admixture with a pharmaceutically-acceptable excipient, which components (a) and (b) are each provided in a form that is suitable for administration in conjunction with the other.

49. A process for the preparation of a compound as defined in claim 27, or a pharmaceutically acceptable salt thereof, comprising the step of reacting a compound of formula II, 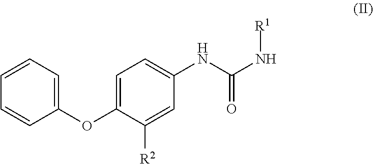
wherein R¹ and R² are as defined in claim 27, with ethoxycarbonyl isocyanate.

50. A compound as claimed in claim 27, wherein the compound is 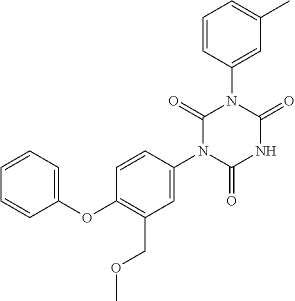 [SMILES: COCc1cc(-n2c(=O)[nH]c(=O)n(-c3cccc(C)c3)c2=O)ccc1Oc1ccccc1]
or a pharmaceutically acceptable salt thereof.

51. A compound as claimed in claim 27, wherein the compound is 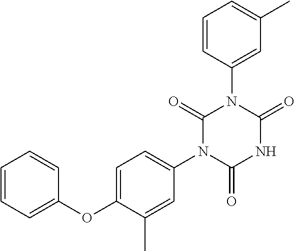 [SMILES: Cc1cccc(-n2c(=O)[nH]c(=O)n(-c3ccc(Oc4ccccc4)c(C)c3)c2=O)c1]
or a pharmaceutically acceptable salt thereof.
